(19) United States
(12) Patent Application Publication
(10) Pub. No.: US 2024/0392332 A1
(43) Pub. Date: Nov. 28, 2024

(54) Cell Free-Based Biocatalyst for Formate Conversion into Value-Added Chemicals

(71) Applicants: Georgia Tech Research Corporation, Atlanta, GA (US); University of Washington, Seattle, WA (US); University of Minnesota, Minneapolis, MN (US); Pacific Northwest National Laboratory, Richland, WA (US)

(72) Inventors: Pamela Peralta-Yahya, Atlanta, GA (US); Alexander S. Beliaev, Richland, WA (US); James M. Carothers, Seattle, WA (US); Shafique Chowdhury, Atlanta, GA (US); Vincent Noireaux, Minneapolis, MN (US)

(21) Appl. No.: 18/675,990

(22) Filed: May 28, 2024

Related U.S. Application Data
(60) Provisional application No. 63/469,224, filed on May 26, 2023.

(51) Int. Cl. C12P 17/18; C12N 9/00; C12N 9/02; C12N 9/06; C12N 9/10; C12N 9/12; C12P 7/40; C12P 13/06

(52) U.S. Cl. CPC C12P 17/182; C12N 9/0014; C12N 9/0028; C12N 9/0051; C12N 9/0093; C12N 9/1014; C12N 9/1241; C12N 9/93; C12P 7/40; C12P 13/06; C12Y 104/04002; C12Y 105/01005; C12Y 108/01004; C12Y 117/01; C12Y 120/01001; C12Y 201/02001; C12Y 201/0201; C12Y 207/07063; C12Y 305/04009; C12Y 603/04003

(57) ABSTRACT

An exemplary embodiment of the present disclosure provides a method of converting formate to a desired compound. The method comprises providing a biocatalyst and formate to form a reaction mixture and reacting at least the biocatalyst with formate to produce a first reaction product.

### CROSS-REFERENCE TO RELATED APPLICATIONS

This application claims the benefit of U.S. Provisional Application Ser. No. 63/469,224, filed on May 26, 2023, which is incorporated herein by reference in its entirety as if fully set forth below.

### GOVERNMENT LICENSE RIGHTS

This invention was made with government support under Agreement No. DE-AR0001514, awarded by the U.S. Department of Energy Advanced Research Project Agency-Energy (ARPA-E) EcoSynBio program, and under Contract DE-AC0576RL01830, awarded by the U.S. Department of Energy. The government has certain rights in the invention.

### SEQUENCE LISTING STATEMENT

This application contains a computer readable Sequence Listing, which has been submitted electronically in XML format and is hereby incorporated by reference in its entirety. Said XML copy, created on May 28, 2024, is named 011529_114553_ST26.xml and is 58,926 bytes bytes in size.

### FIELD OF THE DISCLOSURE

The various embodiments of the present disclosure relate generally to a cell free-based biocatalyst for converting formate into value-added chemicals.

### BACKGROUND

In March 2024, the atmosphere had ˜425 ppm of carbon dioxide (CO₂), a 9% increase since 2010. Increases in CO₂, a greenhouse gas, are associated with rising global temperatures and ocean acidification, negatively impacting human lives and biological systems. Multiple avenues are being explored towards net-zero CO₂ emissions, including mitigating the release of CO₂, directly capturing CO₂ from the environment and storing it in underground geological structures, or using it as a feedstock for chemical production.

Microbes have long been engineered to convert sugars, and more recently, lignocellulosic biomass, into fuels and chemicals. The food versus fuel dilemma limits the expansion of using sugars as a feedstock, while the high cost of lignocellulosic biomass deconstruction limits the economic viability of synthesizing low-cost chemicals from this renewable resource. Biologically upgrading “free” CO₂ into products could enable the economically viable synthesis of fuels and large-volume chemicals. The CO₂ could be from point sources, such as flue gas from steel mills (20-30 mol %), and refineries (30-40 mol %), or could be atmospheric (0.04 vol %) after concentration. Electrons from solar panels or wind farms could be used to electrochemically reduce CO₂ to formate, which now reaches more than 70% Faradaic efficiency, thus making formate a potentially viable substrate at industrial scale. With a solubility of 97.2 g/100 mL, formate is a more biologically accessible form of carbon than CO₂ (0.17 g/100 mL) or bicarbonate (8.2 g/100 mL).

Autotrophic organisms have been engineered to convert CO₂ into value added chemicals, including at commercial scale. For example, LanzaTech uses engineered Clostridia spp. to produce ethanol from steel mill gas. Challenges with engineering organisms that naturally fix CO₂ include 1) slow growth rate (cyanobacteria's growth rate is 5 times slower than Escherichia co/i), 2) low CO₂ fixation rate (cyanobacteria achieves 5 mg/L/h while 10 mg/L/h is needed for industrial applications), and 3) limited engineering of tailoring metabolic pathways to convert central carbon intermediates into value-added chemicals when compared to the biotechnology workhorse chassis Escherichia co/i.

E coli's fast growth rate, extensive synthetic biology tools, and experimental knowledge on the optimization of hundreds of metabolic pathways has made it an attractive chassis to refactor natural and engineered synthetic CO₂ fixation pathways. To date, 4 natural and 12 synthetic formate fixation pathways have been identified, with two of the synthetic pathways having been implemented in microbes. Among them, the low energy (2 ATPs), cofactor (4 NAD(P)Hs), and enzyme (9) requirements of the tetrahydrofolate (THF)-dependent formate fixation/reductive glycine synthesis (THF/rGS) pathway make it the most energetically favorable and succinct pathway to engineer for formate upgrading. Indeed, the THF/rGS pathway has been engineered in E. coli, Saccharomyces cerevisiae and Komagataella phaffi to drive cell growth. Due to the low formate fixation rates, doubling times are slow, (66 hours rather than 30 minutes in the case of E. coli) with limited chemical synthesis observed.

While living organisms must route some of the fixed carbon to cell growth and maintenance, non-living biocatalysts can route 100% of the fixed carbon to chemicals synthesis. Using purified enzyme systems, the artificial starch anabolic pathway, the THF/rGS pathway, the crotonyl-CoA/ethylmalonyl-CoA/hydroxybutyryl-CoA (CETCH) cycle, the tartronyl-CoA pathway and the reductive glyoxylate/pyruvate cycle/malyl-CoA-glycerate (rGPS/MCG) pathway have been constructed. Specifically, the THF/rGS pathway achieved 22% conversion of formate into glycine in the presence of excess formate. Although purified enzyme systems offer exquisite control over the enzyme ratios, the cost involved in multi-enzyme purification will likely limit the scale up of this strategy for large-volume low-cost chemicals.

Unpurified multi-enzyme biocatalysts could route 100% of the fixed carbon to chemical synthesis while keeping the process cost down to enable the economically viable synthesis of industrial chemicals. Such biocatalysts can be generated on demand by direct expression of biosynthetic pathway genes in a nonliving lysate-based CFE, and used without purification for chemical synthesis. Briefly, lysate-based CFEs are composed of microbial cell lysate supplemented with energy compounds and reducing equivalents to support in situ DNA transcription and translation. Previously, individual pathway genes have been overexpressed in E. coli to generate enriched cell lysates, and mixed-and-matched to rapidly prototype biosynthetic pathways to convert glucose into 2,3-butanediol, n-butanol, polyhydroxyalkanoates, and mevalonate with extrapolated biosynthetic productivities (g/L/h) that often surpassed those achieved in living cells. Direct expression of pathway genes in CFE for multi-enzyme biocatalyst generation and use without purification has been applied to the synthesis of n-butanol from glucose by co-expressing 5 genes. A more common strategy, however, has been the individual expression of pathway genes in a different CFE reaction to generate individual biocatalysts followed by mixing them together to establish the pathway. This is the case with the synthesis of 3-hydroxybuterate (2 genes), n-butanol (5 genes), hexanoic acid (5 genes), limonene (9 genes), and azido-sialoglycoproteins (4 genes). In general, CFE-based biocatalysts have relied on the endogenous CFE metabolism to convert glucose into central metabolic intermediates (e.g. acetyl-CoA), regenerate cofactors (NAD(P)H) and energy equivalents (ATP). The only exception is the two-step CFE-based synthesis of styrene from phenylalanine.

### BRIEF SUMMARY

An exemplary embodiment of the present disclosure provides a method of converting formate to a desired compound. The method comprises providing a biocatalyst and formate to form a reaction mixture, and reacting at least the biocatalyst with formate to produce a first reaction product.

In any of the embodiments disclosed herein, the biocatalyst comprises an unpurified mixture of biosynthetic pathway enzymes.

In any of the embodiments disclosed herein, the method can further comprise forming the unpurified mixture of biosynthetic pathway enzymes by a process that involves forming a mixture comprising a cell lysate, one or more biosynthetic pathway genes, one or more cofactors, and one or more energy molecules, and agitating the mixture to allow cell-free expression of the biosynthetic pathway genes to produce the unpurified mixture of biosynthetic pathway enzymes.

In any of the embodiments disclosed herein, the unpurified mixture of biosynthetic pathway enzymes can comprise one or more enzymes selected from the group consisting of formate-tetrahydrofolate ligase (ftl) (SEQ ID NO: 1), methenyltetrahydrofolate cyclohydrolase (fch) (SEQ ID NO: 2), methylenetetrahydrofolate dehydrogenase (mtdA) (SEQ ID NO: 3), glycine cleavage system H protein (gcvH) (SEQ ID NO: 4), glycine cleavage system L protein (gcvL) (SEQ ID NO: 5), glycine cleavage system P protein (gcvP) (SEQ ID NO: 6), glycine cleavage system T protein (gcvT) (SEQ ID NO: 7), lipoate-protein ligase (lplA) (SEQ ID NO: 8), serine hydroxymethyltransferase (shmt) (SEQ ID NO: 9), phosphonate dehydrogenase mutant (ptdh) (SEQ ID NO: 10), formate dehydrogenase (fdh) (SEQ ID NO: 11 or SEQ ID NO:13), and formate dehydrogenase mutant (fdh*) (SEQ ID NO:12).

In any of the embodiments disclosed herein, the unpurified mixture of biosynthetic pathway enzymes are selected from the group consisting of formate-tetrahydrofolate ligase (ftl) (SEQ ID NO: 1), methenyltetrahydrofolate cyclohydrolase (fch) (SEQ ID NO: 2), methylenetetrahydrofolate dehydrogenase (mtdA) (SEQ ID NO: 3), glycine cleavage system H protein (gcvH) (SEQ ID NO: 4), glycine cleavage system L protein (gcvL) (SEQ ID NO: 5), glycine cleavage system P protein (gcvP) (SEQ ID NO: 6), glycine cleavage system T protein (gcvT) (SEQ ID NO: 7), lipoate-protein ligase (lplA) (SEQ ID NO: 8), serine hydroxymethyltransferase (shmt) (SEQ ID NO: 9), phosphonate dehydrogenase mutant (ptdh) (SEQ ID NO: 10), formate dehydrogenase (fdh) (SEQ ID NO: 11 or SEQ ID NO: 13), and formate dehydrogenase mutant (fdh*) (SEQ ID NO: 12).

In any of the embodiments disclosed herein, the reaction mixture can further comprise one or more cofactors and/or one or more energy molecules.

In any of the embodiments disclosed herein, the reaction mixture can further comprise NH₃ and bicarbonate, and the method can further comprise reacting at least the biocatalyst with the NH₃, the bicarbonate, and the first reaction product to produce a second reaction product.

In any of the embodiments disclosed herein, the method can further comprise reacting at least the biocatalyst with the first reaction product and the second reaction product to produce a third reaction product.

In any of the embodiments disclosed herein, the biocatalyst can be in a diluted form.

In any of the embodiments disclosed herein, the first reaction product is 5,10-methylenetetrahydrofolate.

In any of the embodiments disclosed herein, the second reaction product is glycine.

In any of the embodiments disclosed herein, the third reaction product is serine.

In any of the embodiments disclosed herein, the one or more energy molecules is selected from the group consisting of adenosine triphosphate (ATP), guanosine triphosphate (GTP), cytidine triphosphate (CTP), and uridine triphosphate (UTP).

In any of the embodiments disclosed herein, the one or more cofactors is selected from the group consisting of NADH, NADPH, or pyridoxal phosphate (PLP), α-lipoic acid, 1,4-dithiothreitol (DTT), tetrahydrofolate, H₂NaPO₄.

In any of the embodiments disclosed herein, the cell lysate is an E. coli lysate.

In any of the embodiments disclosed herein, the biosynthetic pathway genes can be expressed from one or more plasmids.

In any of the embodiments disclosed herein, the biosynthetic pathway genes can be expressed from linear DNA.

In any of the embodiments disclosed herein, the biosynthetic pathway genes can be expressed from a combination of one or more plasmids and linear DNA.

In any of the embodiments disclosed herein, the formate can be produced by an electrochemical reduction of carbon dioxide.

In any of the embodiments disclosed herein, the method can further comprise reacting at least the biocatalyst with the third reaction product to produce a fourth reaction product, wherein the fourth reaction product is pyruvate.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein.

Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides LC/MS traces of commercial tetrahydrofolate (THF), 5,10-methenyltetrahydrofolate (CH=THF), 5,10 methyleneltetrahydrofolate (CH₂-THF), NADPH, NADP⁺, and NAD⁺ in plain cell-free expression, in accordance with some embodiments of the present disclosure. Chemicals were identified via single ion monitoring at the m/z specified (rt=retention time).

FIGS. 2A-2C provide standard curves using commercial tetrahydrofolate (THF), 5,10-methenyltetrahydrofolate (CH=THF) and 5,10 methyleneltetrahydrofolate (CH₂-THF), in accordance with some embodiments of the present disclosure.

FIGS. 3A-3D provide standard curves using commercial NADH, NAD⁺, NADPH, and NADP⁺, in accordance with some embodiments of the present disclosure.

FIG. 4 provides LC/MS traces of commercial Fmoc-Serine and Fmoc-glycine in plain cell-free expression (CFE), in accordance with some embodiments of the present disclosure. Chemical were identified via extracted ion chromatogram at the m/z specified (rt=retention time).

FIGS. 5A-5B provide standard curves of Fmoc-Serine and Fmoc-Glycine, in accordance with some embodiments of the present disclosure.

FIGS. 6A-6C show the cell-free expression (CFE)-based biocatalyst for the carbon negative synthesis of serine from formate, in accordance with some embodiments of the present disclosure. FIG. 6A provides a schematic of the CFE-based 10-enzyme biocatalyst for the synthesis of serine from formate. FIG. 6B provides the thermodynamics for the synthesis of serine from formate. The ΔG′° of each step was calculated using eQuilibriator (Beber, et al., “eQuilibrator 3.0: a database solution for thermodynamic constant estimation,” Nucleic Acids Res., 50(D1):D603-D609, (2022)) assuming a standard concentration of 1 mM for all reactants. FIG. 6C shows that the CFE-based biocatalyst is independent of endogenous CFE reactions, requires no purification and leverages volumetric expansion to achieve higher product levels. The process consists of three steps: multi-gene expression, biocatalyst dilution (volumetric expansion), and chemical synthesis. Enzyme abbreviations: ftl, formate-tetrahydrofolate ligase; fch, methenyltetrahydrofolate cyclohydrolase; mtdA, methylenetetrahydrofolate dehydrogenase (NADP⁺); gcvHLPT glycine cleavage system H, L, P and T proteins; lplA, lipoate-protein ligase; shmt, serine hydroxymethyltransferase, ptdh*, phosphonate dehydrogenase. Metabolite abbreviations: THF, tetrahydrofolate; CHO-THF, 10-formyltetrahydrofolate; CH=THF, 5,10-methenyltetrahydrofolate; CH₂-THF, 5,10-methylenetetrahydrofolate.

FIGS. 7A-7D show tetrahydrofolate-dependent formate fixation, in accordance with some embodiments of the present disclosure. FIG. 7A provides an overview of the THF-dependent formate fixation module. FIG. 7B shows that the CFE-based ftl+fch biocatalyst converts formate and THF to CH=THF. FIG. 7C shows that the CFE-based mtdA+fdh* biocatalyst reduces CH=THF to CH₂-THF. FIG. 7D shows that the CFE-based Module 1 biocatalyst converts formate and THF to CH=THF and CH₂-THF. For FIGS. 7B-7D, all reactions were done in triplicate. Shown are the means and standard deviations. Enzyme abbreviations: ftl, formate-tetrahydrofolate ligase; fch, methenyltetrahydrofolate cyclohydrolase; mtdA, methylenetetrahydrofolate dehydrogenase; fdh*, formate dehydrogenase (fdh:D227Q/L229H). Metabolite abbreviations: THF, tetrahydrofolate; CH=THF, 5,10-methenyltetrahydrofolate; CH₂-THF, 5,10-methylenetetrahydrofolate.

FIGS. 8A-8B show the synthesis of serine from formate and glycine, in accordance with some embodiments of the present disclosure. FIG. 8A provides an overview of the THF-dependent formate fixation (Module 1) and serine synthesis (Module 3). FIG. 8B provides the percent conversion of glycine to serine by Module, carbon source, NADPH regeneration system, and plasmid number. All reactants were added at stoichiometry. Plasmids were present at 5 nM. Volumetric expansion: 10-fold. All reactions involving mtdA were run semi-anaerobically. All reactions were run in triplicate. Shown are the means and standard deviations. Enzyme abbreviations: ftl, formate-tetrahydrofolate ligase; fch, methenyltetrahydrofolate cyclohydrolase; mtdA, methylenetetrahydrofolate dehydrogenase; ptdh*, phosphonate dehydrogenase mutant; fdh*, formate dehydrogenase mutant; shmt, serine hydroxymethyltransferase. Metabolite abbreviations: THF, tetrahydrofolate; CH=THF, 5,10-methenyltetrahydrofolate; CH₂-THF, 5,10-methylenetetrahydrofolate.

FIGS. 9A-9F show the synthesis of serine and glycine from 5,10-methylenetetrahydrofolate (CH₂-THF), bicarbonate and ammonia, in accordance with some embodiments of the present disclosure. FIG. 9A provides an overview of the reductive glycine module (Module 2) and the serine synthesis module (Module 3). FIG. 9B provides the enzymatic steps involved in reductive glycine synthesis. FIG. 9C provides the western blot showing the protein levels of CFE plasmid DNA of Module 2 genes. FIG. 9D provides the western blot showing the protein levels of CFE linear DNA of Module 2 genes. FIG. 9E provides the western blot showing the protein levels of CFE linear DNA of gcvH and lplA when driven from promoters: P_{T70}, P_{T3} and P_{T7}. FIG. 9F provides the percent conversion of CH₂-THF to serine and glycine by Modules 2+3 using pdth* for NADH regeneration. Unless noted, all reactants were added at stoichiometry. Excess: 10 molar excess of NH₃ and H₂CO₃. Volumetric expansion: 10-fold. All reactions were performed in triplicates. Shown are the means and standard deviations. Abbreviations: gcvL: 50 kDa; lplA: 38 kDa; gcvP: 104 kDa; gcvH: 14 kDa; gcvT: 10 kDa.

FIGS. 10A-10C show the de novo synthesis of serine and glycine from formate, bicarbonate and ammonia, in accordance with some embodiments of the present disclosure.

FIG. 10A shows regeneration of NADH and NADPH by P. stutzeri ptdh* when directly expressed, either independently or in concert, in CFE. FIG. 10B shows de novo synthesis of serine and glycine from formate. Formate, ammonia and bicarbonate were present at stoichiometry in all experiments. 2× mdtA and 2× shmt genes were introduced at 2-fold molar excess to the CFE. 10× less THF: Tetrahydrofolate was added at 10-fold lower concentration than formate. FIG. 10C provides serine and glycine concentration synthesized by Modules 1+2+3 when a 10-fold molar excess of reactants (formate, ammonia and bicarbonate) is added. All reactions were performed under semi-anaerobic conditions and in triplicate. Shown are the means and standard deviations. Enzyme abbreviations: ftl, formate-tetrahydrofolate ligase; fch, methenyltetrahydrofolate cyclohydrolase; mtdA, methylenetetrahydrofolate dehydrogenase; ptdh*, phosphonate dehydrogenase mutant; fdh*, formate dehydrogenase mutant; shmt, serine hydroxymethyltransferase. Metabolite abbreviations: THF, tetrahydrofolate; CH=THF, 5,10-methenyltetrahydrofolate; CH₂-THF, 5,10-methylenetetrahydrofolate.

FIG. 11 provides complete western blots for FIG. 9C.

FIG. 12 provides complete western blots for FIG. 9D.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

In this specification and the appended claims, the singular forms “a,” “an,” and “the” include plural references unless the context clearly dictates otherwise.

The terms “comprising,” “comprises,” and “comprised of” as used herein are synonymous with “including,” “includes,” or “containing,” “contains,” and are inclusive or open-ended and do not exclude additional, non-recited members, elements, or method steps.

The terms “comprising,” “comprises,” and “comprised of” also encompass the term “consisting of” The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of.”

Terms of degree such as “substantially,” “about,” and “approximately” and the symbol “˜” as used herein mean a reasonable amount of deviation of the modified term such that the end result is not significantly changed. These terms of degree should be construed as including a deviation of at least ±0.1% (and up to ±1%, ±5%, or ±10%) of the modified term if this deviation would not negate the meaning of the word it modifies. Unless otherwise clear from context, all numerical values provided herein are modified by the term about. All numerical values provided herein that are modified by terms of degree set forth in this paragraph (e.g., “substantially,” “about,” “approximately,” and “˜”) are also explicitly disclosed without the term of degree. For example, “about 1%” is also explicitly disclosed as “1%”.

The term “and/or” as used herein means that the listed items are present, or used, individually or in combination. In effect, this term means that “at least one of” or “one or more” of the listed items is used or present.

The recitation of numerical ranges by endpoints includes all numbers and fractions subsumed within the respective ranges, as well as the recited endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

Biological systems can directly upgrade carbon dioxide (CO₂) into chemicals. The CO₂ fixation rate of autotrophic organisms, however, is too slow for industrial utility, and the breadth of engineered tailoring pathways for the synthesis of value-added chemicals too limited. Biotechnology workhorse organisms with extensively engineered tailoring pathways have recently been engineered for CO₂ fixation. Yet their low carbon fixation rate, compounded by the fact that living organisms split their carbon between cell growth and chemical synthesis, has led to only cell growth with no chemical synthesis achieved to date. Herein, a lysate-based cell-free expression (CFE) system-based multi-enzyme biocatalyst for the carbon negative de novo synthesis of the industrially relevant amino acids glycine and serine from formate is disclosed. The unpurified 10-enzyme CFE-based biocatalyst leverages tetrahydrofolate (THF)-dependent formate fixation, reductive glycine synthesis, serine synthesis and phosphonate dehydrogenase-dependent NAD(P)H regeneration to convert 39% of formate into serine and glycine, surpassing previous conversions achieved by purified enzyme systems. Correlating the concentration of linear DNA added to the CFE reactions to the levels of protein synthesis achieved allowed the identification of optimal gene ratios to achieve maximal formate conversion. Efficient THF recycling enabled 10-fold lower cofactor loading to reach similar (32%) formate to serine and glycine conversion, reducing the cost of the process. Towards the scale up of CFE-based processes, the CFE-based multi-enzyme catalyst can be diluted up to 200-fold using inexpensive buffer while retaining catalytic activity. Such volumetric expansion enabled greater substrate loading, leading to higher levels of synthesized products using the same CFE inputs. As formate can be directly obtained from CO₂ via electrochemical reduction, the carbon-negative de novo synthesis of serine from formate opens the door to the future synthesis pyruvate and a wide array of chemicals from CO₂.

A CFE-based multi-enzyme biocatalyst for use without purification for the carbon negative de novo synthesis of serine and glycine from formate (Figure TA) is disclosed herein. Serine, an industrial chemical and animal feed, has an annual global production of 350 MT/year with fermentation being the preferred production process (Wendisch, “Metabolic Engineering Advances and Prospects for Amino Acid Production,” Metab Eng 58:17-34 (2020)). Glycine is a building block for the synthesis of a variety of chemicals, including herbicides and insecticides and has an annual global production of 22,000 MT/year (Wendisch, “Metabolic Engineering Advances and Prospects for Amino Acid Production,” Metab Eng 58:17-34 (2020)). Specifically, a lysate-based E. coli CFE is used to express a 10-gene biosynthetic pathway composed of THF-dependent formate fixation (Module 1), reductive glycine synthesis (Module 2) and serine synthesis (Module 3). An engineered bifunctional phosphonate-dependent NAD(P)H regeneration system supports high co-factor concentration, driving reactions that are close to thermodynamic equilibrium forward and enables use of formate exclusively as a carbon source. Correlating the concentration of pathway genes added to the CFE with the protein synthesis levels achieved was pivotal to optimizing the conversion of formate to glycine and serine. Finally, volumetric expansion of the CFE-based biocatalyst with inexpensive buffer enabled greater feedstock loading and increased chemical synthesis levels using the same CFE inputs, which will be pivotal in the scale-up of cell-free systems to produce large-volume chemicals. Overall, the CFE-based biocatalyst achieved a 39% combined conversion of formate to glycine and serine. To Applicant's knowledge, this is the first carbon negative de novo synthesis of a chemical from formate using a lysate-based CFE-based biocatalyst, which does not require purification before use. The CFE-based biocatalyst surpasses the 22% carbon conversion achieved by the rGS pathway using a purified enzyme system (Wu et al., “Enzymatic Electrosynthesis of Glycine from CO₂ and NH₃,” Angewandte Chemie, 135:e202218387 (2023)) and the engineered rGS pathway in E. coli where the output was cell growth. Looking ahead, the pathway could be extended beyond serine to pyruvate, a key intermediate to access a variety of chemicals from aromatics and terpenes to alcohols and polymers.

An exemplary embodiment of the present disclosure provides a method of converting formate to a desired compound. The method comprises providing a biocatalyst and formate to form a reaction mixture and reacting at least the biocatalyst with formate to produce a first reaction product.

In some embodiments, the biocatalyst comprises an unpurified mixture of biosynthetic pathway enzymes. Exemplary biosynthetic pathway enzymes include formate-tetrahydrofolate ligase (ftl) (SEQ ID NO: 1), methenyltetrahydrofolate cyclohydrolase (fch) (SEQ ID NO: 2), methylenetetrahydrofolate dehydrogenase (mtdA) (SEQ ID NO: 3), glycine cleavage system H protein (gcvH) (SEQ ID NO: 4), glycine cleavage system L protein (gcvL) (SEQ ID NO: 5), glycine cleavage system P protein (gcvP) (SEQ ID NO: 6), glycine cleavage system T protein (gcvT) (SEQ ID NO: 7), lipoate-protein ligase (lplA) (SEQ ID NO: 8), serine hydroxymethyltransferase (shmt) (SEQ ID NO: 9), phosphonate dehydrogenase mutant (ptdh) (SEQ ID NO: 10), formate dehydrogenase (fdh) (SEQ ID NO: 11 or SEQ ID NO: 13), and formate dehydrogenase mutant (fdh*) (SEQ ID NO: 12). In some embodiments, the unpurified mixture of biosynthetic pathway enzymes comprises about 1 to about 35 enzymes. In some embodiments, the unpurified mixture of biosynthetic pathway enzymes comprises any number or range of enzymes between 1 and 35 enzymes. For example, in some embodiments, the unpurified mixture of biosynthetic pathway enzymes comprises 1, 2, 3, 4, 5, 8, 13, 18, 22, 33, about 1 to about 5, about 1 to about 10, about 1 to about 15, about 1 to about 20, about 1 to about 25, about 1 to about 30, about 1 to about 35, about 5 to about 10, about 5 to about 15, about 5 to about 20, about 5 to about 25, or about 5 to about 30, about 5 to about 35, about 10 to about 15, about 10 to about 20, about 10 to about 25, about 10 to about 30, about 10 to about 35, about 15 to about 20, about 15 to about 25, about 15 to about 35, about 20 to about 25, about 20 to about 30, about 20 to about 35, about 25 to about 30, about 25 to about 35, or about 30 to about 35 enzymes.

In some embodiments, the method can further comprise forming the unpurified mixture of biosynthetic pathway enzymes by a process that involves forming a mixture comprising a cell lysate, one or more biosynthetic pathway genes, one or more cofactors, and one or more energy molecules, and agitating the mixture to allow cell-free expression of the biosynthetic pathway genes to produce the unpurified mixture of biosynthetic pathway enzymes. Exemplary biosynthetic pathway genes include ftl (SEQ ID NO: 14), fch (SEQ ID NO: 15), mtdA (SEQ ID NO: 16), gcvH (SEQ ID NO: 17), gcvL (SEQ ID NO: 18), gcvP (SEQ ID NO: 19), gcvT (SEQ ID NO: 20), lplA (SEQ ID NO: 21), shmt (SEQ ID NO: 22), ptdh* (SEQ ID NO: 23), fdh (SEQ ID NO: 24 or SEQ ID NO: 26), and fdh* (SEQ ID NO: 25). In some embodiments the gene is optimized for efficient translation in E. coli by modifying the DNA sequence. Exemplary modifications include replacing codons with those often used by E. coli, testing RNA folding, and changing codons manually to optimize folding.

Cell-free expression is a method that enables in vitro protein synthesis through the expression of natural or synthetic DNA. In this process, the molecular components necessary for transcription and translation are isolated from microbial cells by preparing a cell lysate stripped of genetic material and membranes. The lysate is supplemented with the necessary energy compounds and cofactors to support DNA transcription and translation. As disclosed herein, Cell-free expression is used for the direct expression of biosynthetic pathway genes to generate a multi-enzyme biocatalyst, which can be used without purification and applied to the synthesis of desired compounds from formate.

In some embodiments, the reaction mixture can further comprise one or more cofactors and/or one or more energy molecules. For example, in some embodiments, the one or more energy molecules is selected from the group consisting of adenosine triphosphate (ATP), guanosine triphosphate (GTP), cytidine triphosphate (CTP), and uridine triphosphate (UTP). In some embodiments, the one or more cofactors is selected from the group consisting of NADH, NADPH, or pyridoxal phosphate (PLP), α-lipoic acid, 1,4-dithiothreitol (DTT), tetrahydrofolate, H₂NaPO₄.

In some embodiments, the reaction mixture can further comprise NH₃ and bicarbonate, and the method can further comprise reacting at least the biocatalyst with the NH₃, the bicarbonate, and the first reaction product to produce a second reaction product. As used herein, “bicarbonate” refers to the bicarbonate ion (HCO₃⁻), which can be used in various forms, including but not limited to carbonic acid, sodium bicarbonate, potassium bicarbonate, and ammonium bicarbonate. In some embodiments, ammonium bicarbonate is the source of both the bicarbonate ion and the ammonia.

In some embodiments, the method can further comprise reacting at least the biocatalyst with the first reaction product and the second reaction product to produce a third reaction product. In some embodiments, the first reaction product is 5,10-methylenetetrahydrofolate. In some embodiments, the second reaction product is glycine. In some embodiments, the third reaction product is serine. In some embodiments, the method can further comprise reacting at least the biocatalyst with the third reaction product to produce a fourth reaction product, wherein the fourth reaction product is pyruvate. To produce pyruvate, the unpurified mixture of biosynthetic pathway enzymes can include serine dehydratase (EC 4.3.1.17) in addition to the enzymes disclosed above to produce serine. To include serine dehydratase in the unpurified mixture of biosynthetic pathway enzymes, the gene that codes for serine dehydratase can be included in the cell-free expression to form the unpurified mixture of biosynthetic pathway enzymes.

In some embodiments, the cell lysate is an E. coli lysate.

In some embodiments, the biosynthetic pathway genes can be expressed from one or more plasmids. In other embodiments, the biosynthetic pathway genes can be expressed from linear DNA. In other embodiments, the biosynthetic pathway genes can be expressed from a combination of one or more plasmids and linear DNA.

In some embodiments, the formate can be produced by the reduction of carbon dioxide. Accordingly, in some embodiments, the method can further comprise obtaining formate from carbon dioxide. For example, carbon dioxide can be converted to formate via electrochemical reduction, photochemical reduction, photoelectrochemical reduction, or hydrogenation. In some embodiments, solar panels or wind farms can be used to electrochemically reduce CO₂ to formate. In some embodiments, CO₂ can be obtained from point sources, such as flue gas from steel mills and refineries, or can be atmospheric. In another embodiment, the unpurified mixture of biosynthetic pathway enzymes can include an enzyme, such as formate dehydrogenase, that catalyzes the conversion of carbon dioxide to formate.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

### EXAMPLES

The following Examples are presented to illustrate various aspects of the present disclosure, but are by no means intended to limit its scope.

### Example 1—Materials and Methods

### Materials

All materials, including chemicals, solvents, kits, plasmids, primers, protein sequences and gene sequences can be found in the Tables 1-8. Sources for key substrates, co-factors, and products: Tetrahydrofolate, 5,10-methenyl THF, 5,10-methylene THF, NADH, and NADPH were purchased from Cayman Chemicals. Formic acid was purchased from Fischer Scientific. Serine, glycine, ammonia solution in water, ATP, DTT, u-lipoic acid, catechol, sodium dihydrogen phosphate and sodium bicarbonate were purchased from Millipore Sigma. Pyridoxal-5-phosphate was purchased from TCI chemicals. Fmoc chloride was purchased from Oakwood chemical. Cell-free expression system was purchased from Arbor Biosciences.

TABLE 1
Table of Reagents.

| Reagents | Vendor | Catalog# |
|---|---|---|
| 1,4-dithiothreitol (DTT) | Sigma | 12/3/3483 |
| 25% ammonia in water | Millipore Sigma | 1.05422 |
| 5,10 methylene tetrahydrofolate | Cayman Chemicals | 33967 |
| 5,10-methenyl tetrahydrofolate | Cayman Chemicals | 31333 |
| ATP | Millipore Sigma | A6419 |
| catechol | Millipore Sigma | PHL823720 |
| Fmoc Chloride | Oakwood Chemical | 22072 |
| Formic acid | Fischer scientific | A117-50 |
| Glycine | Millipore Sigma | 07126 |
| NADH | Cayman Chemicals | 16078 |
| NADPH | Cayman Chemicals | 9000743 |
| Pyridoxal-5-phosphate | TCI chemicals | C0377 |
| Serine | Millipore Sigma | S4500 |
| Sodium bicarbonate | Millipore Sigma | S5761 |
| Sodium dihydrogen phosphate | Millipore Sigma | 1.0637 |
| Tetrahydrofolate | Cayman Chemicals | 18263 |
| u-lipoic acid | Millipore Sigma | 1368301 |
| NuPAGE ™ 4 to 12%, | Invitrogen | NP0329BOX |
| Bis-Tris, 1.0-1.5 mm, | | |
| Mini Protein Gels | | |
| NuPAGE ™ LDS | Invitrogen | NP0007 |
| Sample Buffer (4X) | | |
| NuPAGE ™ MES SDS | Invitrogen | NP0002 |
| Running Buffer (20X) | | |
| PageRuler prestained protein ladder | Thermo Scientific | 26616 |
| Green Fluorescent Protein | Millipore Sigma | 14-392 |
| iBlot ™ Transfer Stack, | Invitrogen | IB301002 |
| nitrocellulose, mini | | |
| Monoclonal | Millipore Sigma | H1029 |
| Anti-polyHistidine antibody | | |
| produced in mouse | | |
| Anti-Mouse IgG | Millipore Sigma | A3688 |
| (whole molecule)-Alkaline | | |
| Phosphatase antibody | | |
| produced in goat | | |

TABLE 2
Table of Solvents.

| Reagents | Vendor | Catalog# |
|---|---|---|
| Acetic acid | EMD Millipore | 101830 |
| Methanol | Fischer | A452-4 |
| | Scientific | |
| Tributylamine | Sigma | 90780 |
| Ethy Acetate | Sigma | 319902 |
| Acetone | Fischer | 326801000 |
| | Scientific | |

TABLE 3
Table of Kits

| Kit | Vendor | Catalog # |
|---|---|---|
| myTXTL Sigma 70 mastr mix | Arbor | 507096 |
| | Biosciences | |
| | Arbor | |
| CFE linear DNA kit | Biosciences | 508096 |
| XCell SureLock ™ Mini Cell | Invitrogen | EI0001 |

### Plasmid DNA Formate to Serine Biosynthetic Pathway Construction

M. extorquens ftl, fch, and mtdA, A. thaliana fdh, and fdh* (fdh:D227Q/L229H)44 were codon optimized for E. coli. The E. coli genes gcvHLPT, lplA, and shmt, as well as P. stutzeri ptdh*46 were used without optimization. All sequences used in this work can be found in Tables 4-7.

TABLE 4
Table of enzymes

| Origin | Enzyme | Sequence |
|---|---|---|
| Methylobacterium | formate- | MPSDIEIARAATLKPIAQVAEKLGIPDEALHNYGKHIAKIDHDF |
| extorquens | tetrahydrofolate ligase | IASLEGKPEGKLVLVTAISPTPAGEGKTTTTVGLGDALNRIGKR |
| | (SEQ ID NO: 1) | AVMCLREPSLGPCFGMKGGAAGGGKAQVVPMEQINLHFTGDFHA |
| | | ITSAHSLAAALIDNHIYWANELNIDVRRIHWRRVVDMNDRALRA |
| | | INQSLGGVANGFPREDGFDITVASEVMAVECLAKNLADLEERLG |
| | | RIVIAETRDRKPVTLADVKATGAMTVLLKDALQPNLVQTLEGNP |
| | | ALIHGGPFANIAHGCNSVIATRTGLRLADYTVTEAGFGADLGAE |
| | | KFIDIKCRQTGLKPSAVVIVATIRALKMHGGVNKKDLQAENLDA |
| | | LEKGFANLERHVHNVRSFGLPVVVGVNHFFQDTDAEHVRLKELC |
| | | RDRLQVEAITCKHWAEGGAGAEALAQAVVKLAEGEQKPLTFAYE |
| | | TETKITDKIKAIATKLYGAADIQIESKAATKLAGFEKDGYGKLP |
| | | VCMAKTQYSFSTDPTLMGAPSGHLVSVRDVRLSAGAGFVVVICG |
| | | EIMTMPGLPKVPAADTIRLDANGQIDGLF |
| | methenyl- | MAGNETIETFLDGLASSAPTPGGGGAAAISGAMGAALVSMVCNL |
| | tetrahydrofolate | TIGKKKYVEVEADLKQVLEKSEGLRRTLTGMIADDVEAFDAVMG |
| | cyclohydrolase (SEQ | AYGLPKNTDEEKAARAAKIQEALKTATDVPLACCRVCREVIDLA |
| | ID NO: 2) | EIVAEKGNLNVISDAGVAVLSAYAGLRSAALNVYVNAKGLDDRA |
| | | FAEERLKELEGLLAEAGALNERIYETVKSKVN |
| | methylenetetrahydrofol | MSKKLLFQFDTDATPSVFDVVVGYDGGADHITGYGNVTPDNVGA |
| | ate dehydrogenase | YVDGTIYTRGGKEKQSTAIFVGGGDMAAGERVFEAVKKRFFGPF |
| | (SEQ ID NO: 3) | RVSCMLDSNGSNTTAAAGVALVVKAAGGSVKGKKAVVLAGTGPV |
| | | GMRSAALLAGEGAEVVLCGRKLDKAQAAADSVNKRFKVNVTAAE |
| | | TADDASRAEAVKGAHFVFTAGAIGLELLPQAAWQNESSIEIVAD |
| | | YNAQPPLGIGGIDATDKGKEYGGKRAFGALGIGGLKLKLHRACI |
| | | AKLFESSEGVEDAEEIYKLAKEMA |
| Escherichia | glycine cleavage | MSNVPAELKYSKEHEWLRKEADGTYTVGITEHAQELLGDMVEVD |
| coli | system (gcv) Hprotein | LPEVGATVSAGDDCAVAESVKAASDIYAPVSGEIVAVNDALSDS |
| | (SEQ ID NO: 4) | PELVNSEPYAGGWIFKIKASDESELESLLDATAYEALLEDE |
| | glycine cleavage | MSTEIKTQVVVLGAGPAGYSAAFRCADLGLETVIVERYNTLGGV |
| | system (gcv) Lprotein | CLNVGCIPSKALLHVAKVIEEAKALAEHGIVFGEPKTDIDKIRT |
| | (SEQ ID NO: 5) | WKEKVINQLTGGLAGMAKGRKVKVVNGLGKFTGANTLEVEGENG |
| | | KTVINFDNAIIAAGSRPIQLPFIPHEDPRIWDSTDALELKEVPE |
| | | RLLVMGGGIIGLEMGTVYHALGSQIDVVEMFDQVIPAADKDIVK |
| | | VFTKRISKKFNLMLETKVTAVEAKEDGIYVTMEGKKAPAEPQRY |
| | | DAVLVAIGRVPNGKNLDAGKAGVEVDDRGFIRVDKQLRTNVPHI |
| | | FAIGDIVGQPMLAHKGVHEGHVAAEVIAGKKHYFDPKVIPSIAY |
| | | TEPEVAWVGLTEKEAKEKGISYETATFPWAASGRAIASDCADGM |
| | | TKLIFDKESHRVIGGAIVGTNGGELLGEIGLAIEMGCDAEDIAL |
| | | TIHAHPTLHESVGLAAEVFEGSITDLPNPKAKKK |
| | glycine cleavage | MTQTLSQLENSGAFIERHIGPDAAQQQEMLNAVGAQSLNALTGQ |
| | system (gcv) P protein | IVPKDIQLATPPQVGAPATEYAALAELKAIASRNKRFTSYIGMG |
| | (SEQ ID NO: 6) | YTAVQLPPVILRNMLENPGWYTAYTPYQPEVSQGRLEALLNFQQ |
| | | VTLDLTGLDMASASLLDEATAAAEAMAMAKRVSKLKNANRFFVA |
| | | SDVHPQTLDVVRTRAETFGFEVIVDDAQKVLDHQDVFGVLLQQV |
| | | GTTGEIHDYTALISELKSRKIVVSVAADIMALVLLTAPGKQGAD |
| | | IVFGSAQRFGVPMGYGGPHAAFFAAKDEYKRSMPGRIIGVSKDA |
| | | AGNTALRMAMQTREQHIRREKANSNICTSQVLLANIASLYAVYH |
| | | GPVGLKRIANRIHRLTDILAAGLQQKGLKLRHAHYFDTLCVEVA |
| | | DKAGVLTRAEAAEINLRSDILNAVGITLDETTTRENVMQLENVL |
| | | LGDNHGLDIDTLDKDVAHDSRSIQPAMLRDDEILTHPVENRYHS |
| | | ETEMMRYMHSLERKDLALNQAMIPLGSCTMKLNAAAEMIPITWP |
| | | EFAELHPFCPPEQAEGYQQMIAQLADWLVKLTGYDAVCMQPNSG |
| | | AQGEYAGLLAIRHYHESRNEGHRDICLIPASAHGTNPASAHMAG |
| | | MQVVVVACDKNGNIDLTDLRAKAEQAGDNLSCIMVTYPSTHGVY |
| | | EETIREVCEVVHQFGGQVYLDGANMNAQVGITSPGFIGADVSHL |
| | | NLHKTFCIPHGGGGPGMGPIGVKAHLAPFVPGHSVVQIEGMLTR |
| | | QGAVSAAPFGSASILPISWMYIRMMGAEGLKKASQVAILNANYI |
| | | ASRLQDAFPVLYTGRDGRVAHECILDIRPLKEETGISELDIAKR |
| | | LIDYGFHAPTMSFPVAGTLMVEPTESESKVELDRFIDAMLAIRA |
| | | EIDQVKAGVWPLEDNPLVNAPHIQSELVAEWAHPYSREVAVEPA |
| | | GVADKYWPTVKRLDDVYGDRNLFCSCVPISEYQ |
| | glycine cleavage | MAQQTPLYEQHTLCGARMVDFHGWMMPLHYGSQIDEHHAVRTDA |
| | system (gcv) Tprotein | GMFDVSHMTIVDLRGSRTREFLRYLLANDVAKLTKSGKALYSGM |
| | (SEQ ID NO: 7) | LNASGGVIDDLIVYYFTEDFFRLVVNSATREKDLSWITQHAEPF |
| | | GIEITVRDDLSMIAVQGPNAQAKAATLENDAQRQAVEGMKPFFG |
| | | VQAGDLFIATTGYTGEAGYEIALPNEKAADFWRALVEAGVKPCG |
| | | LGARDTLRLEAGMNLYGQEMDETISPLAANMGWTIAWEPADRDE |
| | | IGREALEVQREHGTEKLVGLVMTEKGVLRNELPVRFTDAQGNQH |
| | | EGIITSGTESPTLGYSIALARVPEGIGETAIVQIRNREMPVKVT |
| | | KPVFVRNGKAVA |
| | lipoate-protein ligase | MSTLRLLISDSYDPWENLAVEECIFRQMPATQRVLELWRNADTV |
| | (SEQ ID NO: 8) | VIGRAQNPWKECNTRRMEEDNVRLARRSSGGGAVFHDLGNTCFT |
| | | FMAGKPEYDKTISTSIVLNALNALGVSAEASGRNDLVVKTVEGD |
| | | RKVSGSAYRETKDRGFHHGTLLLNADLSRLANYLNPDKKKLAAK |
| | | GITSVRSRVTNLTELLPGITHEQVCEAITEAFFAHYGERVEAEI |
| | | ISPNKTPDLPNFAETFARQSSWEWNFGQAPAFSHLLDERFTWGG |
| | | VELHFDVEKGHITRAQVFTDSLNPAPLEALAGRLQGCLYRADML |
| | | QQECEALLVDFPEQEKELRELSAWMAGAVR |
| | serine | MLKREMNIADYDAELWQAMEQEKVRQEEHIELIASENYTSPRVM |
| | hydroxymethyltransfer | QAQGSQLTNKYAEGYPGKRYYGGCEYVDIVEQLAIDRAKELFGA |
| | ase (SEQ ID NO: 9) | DYANVQPHSGSQANFAVYTALLEPGDTVLGMNLAHGGHLTHGSP |
| | | VNFSGKLYNIVPYGIDATGHIDYADLEKQAKEHKPKMIIGGFSA |
| | | YSGVVDWAKMREIADSIGAYLFVDMAHVAGLVAAGVYPNPVPHA |
| | | HVVTTTTHKTLAGPRGGLILAKGGSEELYKKLNSAVFPGGQGGP |
| | | LMHVIAGKAVALKEAMEPEFKTYQQQVAKNAKAMVEVFLERGYK |
| | | VVSGGTDNHLFLVDLVDKNLTGKEADAALGRANITVNKNSVPND |
| | | PKSPFVTSGIRVGTPAITRRGFKEAEAKELAGWMCDVLDSINDE |
| | | AVIERIKGKVLDICARYPVYA |
| Pseudomonas | phosphonate | MLPKLVITHRVHEEILQLLAPHCELITNQTDSTLTREEILRRCR |
| stutzeri | dehydrogenase mutant | DAQAMMAFMPDRVDADFLQACPELRVIGCALKGFDNEDVDACTA |
| | (SEQ ID NO: 10) | RGVWLTFVPDLLTVPTAELAIGLAVGLGRHLRAADAFVRSGKER |
| | | GWQPRFYGTGLDNATVGFLGMGAIGLAMADRLQGWGATLQYHAA |
| | | KALDTQTEQRLGLRQVACSELFASSDFILLALPLNADTLHLVNA |
| | | ELLALVRPGALLVNPCRGSVVDEAAVLAALERGQLGGYAADVFE |
| | | MEDWARADRPQQIDPALLAHPNTLFTPHIGSAVRAVRLEIERCA |
| | | AQNILQALAGERPINAVNRLPKAEPAAC |
| Arabidopsis | formate dehydrogenase | MAMRQAAKATIRACSSSSSSGYFARRQFNASSGDSKKIVGVFYK |
| | (SEQ ID NO: 11) | ANEYATKNPNFLGCVENALGIRDWLESQGHQYIVTDDKEGPDCE |
| | | LEKHIPDLHVLISTPFHPAYVTAERIKKAKNLKLLLTAGIGSDH |
| | | IDLQAAAAAGLTVAEVTGSNVVSVAEDELMRILILMRNFVPGYN |
| | | QVVKGEWNVAGIAYRAYDLEGKTIGTVGAGRIGKLLLQRLKPFG |
| | | CNLLYHDRLQMAPELEKETGAKFVEDLNEMLPKCDVIVINMPLT |
| | | EKTRGMENKELIGKLKKGVLIVNNARGAIMERQAVVDAVESGHI |
| | | G |
| | formate dehydrogenase | MRQAAKATIRACSSSSSSGYFARRQFNASSGDSKKIVGVFYKAN |
| | mutant (SEQ ID NO: | EYATKNPNELGCVENALGIRDWLESQGHQYIVTDDKEGPDCELE |
| | 12) | KHIPDLHVLISTPFHPAYVTAERIKKAKNLKLLLTAGIGSDHID |
| | | LQAAAAAGLTVAEVTGSNVVSVAEDELMRILILMRNFVPGYNQV |
| | | VKGEWNVAGIAYRAYDLEGKTIGTVGAGRIGKLLLQRLKPFGCN |
| | | LLYHQRHQMAPELEKETGAKFVEDLNEMLPKCDVIVINMPLTEK |
| | | TRGMENKELIGKLKKGVLIVNNARGAIMERQAVVDAVESGHIG |
| Candida | formate dehydrogenase | MKIVLVLYDAGKHAADEEKLYGCTENKLGIANWLKDQGHELITT |
| boidinii | (SEQ ID NO: 13) | SDKEGGNSVLDQHIPDADIIITTPFHPAYITKERIDKAKKLKLV |
| | | VVAGVGSDHIDLDYINQTGKKISVLEVTGSNVVSVAEHVLMTML |
| | | VLVRNFVPAHEQIINHDWEVAAIAKDAYDIEGKTIATIGAGRIG |
| | | YRVLERLVPENPKELLYYDYQALPKDAEEKVGARRVENIEELVA |
| | | QADIVTINAPLHAGTKGLINKELLSKFKKGAWLVNTARGAICVA |
| | | EDVAAALESGQLRGYGGDVWFPQPAPKDHPWRDMRNKYGAGNAM |
| | | TPHYSGTTLDAQTRYAEGTKNILESFFTGKFDYRPQDIILLNGE |
| | | YITKAYGKHDKK |

TABLE 5
Table of primers

| Primer Name | Sequence |
|---|---|
| SC12 (SEQ ID NO: 27) | GCGGTGATAATGGTTGCAG |
| JS4 (SEQ ID NO: 28) | ACTGGGTTGAAGGCTCTCAA |
| RW9 (SEQ ID NO: 29) | GACTATCGCACCATCAGC |
| RW10 (SEQ ID NO: 30) | CTGTCCTACGAGTTGCATG |
| GH1 (SEQ ID NO: 31) | GTGATGTCGGCGATATAGGC |
| GH2 (SEQ ID NO: 32) | CTGTCCGACCGCTTTG |
| GH3 (SEQ ID NO: 33) | CGCCTGATGCGTGAAC |
| GH4 (SEQ ID NO: 34) | GTAGCACCTGAAGTCAGCC |

TABLE 6
Table of promoters

| Promoter | Sequence |
|---|---|
| P_{T70} (SEQ | TGAGCTAACACCGTGCGTGTTGACAATTTTACCTCTGG |
| ID NO: 35) | CGGTGATAATGGTTGCA |
| P_{T3} (SEQ | ATTAACCCTCACTAAAGGG |
| ID NO: 36) | |

TABLE 7
Sequences of genes evaluated

| Origin | Gene | Enzyme | Notes | Sequences Used |
|---|---|---|---|---|
| Methylobacterium | fil | formate- | Q83WS0 | atgccgagcgatattgaaattgcacgcgctgct |
| extorquens | (SEQ ID | THF | (optimized) | actctgaaaccgattgcgcaagttgcggagaaa |
| | NO: 14) | ligase | | ctgggtattccggacgaggctcttcataattat |
| | | | | ggcaaacatatcgctaaaatcgaccatgacttt |
| | | | | attgcttctcttgagggtaaaccagagggcaaa |
| | | | | cttgttctggttactgctatttcgccgactcca |
| | | | | gctggcgagggcaaaactactactactgttggt |
| | | | | ctgggcgatgctctcaaccgcattggcaaacgt |
| | | | | gctgttatgtgtctgcgcgagccctctctcggc |
| | | | | ccctgttttggcatgaaaggcggcgctgctggt |
| | | | | ggcggcaaagctcaggttgttccgatggagcag |
| | | | | attaatctgcacttcaccggcgattttcacgct |
| | | | | attacttctgctcactctctcgctgctgctctg |
| | | | | attgataaccatatttattgggctaacgaactg |
| | | | | aatattgacgttcgccgcattcattggcgccgc |
| | | | | gttgttgatatgaacgatcgggctctgcgcgct |
| | | | | attaatcagtctctcggcggcgttgctaatggc |
| | | | | tttccgcgcgaggatgggtttgacattactgtt |
| | | | | gcttctgaggttatggctgtgttttgcctcgcc |
| | | | | aagaatctggctgatcttgaggagcggctcggc |
| | | | | cgcattgttattgcagaaactcgcgatcgcaaa |
| | | | | ccggttactctggctgatgttaaagctactggc |
| | | | | gctatgactgttctgctcaaggatgctcttcag |
| | | | | ccgaatctcgtgcagactctggagggcaacccg |
| | | | | gctctgattcacggcggcccgtttgctaacatt |
| | | | | gctcatggctgtaactcggttattgctactcgc |
| | | | | actggcctgcggctcgctgactatactgttact |
| | | | | gaggctggctttggcgctgatctcggcgctgag |
| | | | | aaattcattgatattaaatgtcgccagactggc |
| | | | | ctcaagccctctgctgttgttattgttgctacg |
| | | | | attcgcgctctcaaaatgcatggcggcgttaac |
| | | | | aagaaagatctccaggctgagaatctggatgcg |
| | | | | ctggagaaaggttttgcaaatcttgagcgccat |
| | | | | gttcacaatgttcgctcttttggcctgccggtt |
| | | | | gttgttggtgttaaccacttctttcaggatact |
| | | | | gatgctgagcatgttcggttgaaagaactgtgc |
| | | | | cgcgatcggcttcaggttgaggctattacttgt |
| | | | | aagcattgggctgagggcggcgcaggcgcagaa |
| | | | | gcactggcacaggcagttgttaaactggctgaa |
| | | | | ggcgagcagaaaccgctgacttttgcatatgag |
| | | | | accgaaactaagattactgacaagattaaggca |
| | | | | attgctactaaactgtatggtgctgctgatatt |
| | | | | cagattgagtctaaagccgccactaagctcgct |
| | | | | ggcttcgagaaagatggctatggtaagctgccg |
| | | | | gtctgtatggccaagactcaatattcattttct |
| | | | | actgatccgactcttatgggcgctccctctggt |
| | | | | catctggtttctgtgcgcgatgttcgcctctct |
| | | | | gctggcgctggcttcgttgttgttatttgtggt |
| | | | | gagattatgaccatgccgggtctgccgaaggtt |
| | | | | ccagcagcagatactattcgcctcgatgctaac |
| | | | | ggtcagattgatgggctgttctag |
| | fch | methenyl- | Q49145 | atggctggcaatgagactattgaaacattcttg |
| | (SEQ ID | THF | (optimized) | gacggcctggcatcatctgctccgactcccggc |
| | NO: 15) | cyclohydrolase | | ggcggcggtgcagcagcaatttctggcgcaatg |
| | | | | ggcgcagcacttgtttctatggtttgcaatctt |
| | | | | actattggcaagaagaaatatgttgaggttgag |
| | | | | gcagacttaaaacaggttctggagaaatctgaa |
| | | | | ggcctgcgccgcactctcactggcatgattgca |
| | | | | gacgacgttgaagcctttgacgcagttatgggc |
| | | | | gcttatgggctgccgaagaatactgacgaagag |
| | | | | aaagcagcacgcgcagcaaagattcaagaggca |
| | | | | ctcaaaactgcaactgacgttccgctcgcatgt |
| | | | | tgtcgcgtttgtcgcgaggttattgatctggca |
| | | | | gagattgttgcagagaaaggcaatctcaatgtt |
| | | | | atttctgatgcaggcgttgcagtgctctctgct |
| | | | | tatgcaggtctgcgctctgctgcacttaatgtc |
| | | | | tatgtaaatgcaaaaggcctcgacgaccgcgca |
| | | | | tttgcagaggagcggcttaaagagctggagggc |
| | | | | ctactggctgaggcaggtgcactcaatgagcga |
| | | | | atttatgagactgttaaatctaaagtgaattga |
| | mtdA | methylene | P55818 | atgtctaagaaactgctctttcagtttgacact |
| | (SEQ ID | THF | (optimized) | gatgcaactccgtctgtatttgacgttgttgtt |
| | NO: 16) | dehydrogenase | | ggctatgacggcggtgcagaccatattactggc |
| | | | | tatggcaatgttactcccgacaatgttggcgca |
| | | | | tatgttgacggcactatttatactcgtggaggc |
| | | | | aaagagaaacagtctacagcaatctttgttggc |
| | | | | ggcggcgacatggcagcaggcgagcgggtattt |
| | | | | gaggcagtaaagaagcgtttctttggcccgttt |
| | | | | cgcgtttcttgtatgctggattctaatggctct |
| | | | | aatactactgcagcagcaggcgttgcactcgtt |
| | | | | gttaaagcagcaggcggctctgttaaaggcaag |
| | | | | aaagcagttgttctcgcaggtactggtccggtt |
| | | | | ggtatgcgctctgcagctctgttagccggcgag |
| | | | | ggcgcagaggttgttctgtgtgggcgcaaactc |
| | | | | gacaaagcacaggcagcagcagattctgttaat |
| | | | | aaacgcttcaaagttaatgttactgcagcagag |
| | | | | actgcagacgacgcatctcgcgcagaggccgtg |
| | | | | aaaggcgcacattttgtctttactgcaggtgca |
| | | | | attggccttgaactgctgccgcaggcagcatgg |
| | | | | cagaatgagtcttctattgaaattgtggccgat |
| | | | | tataatgcacagccgccgctcggcattggcggg |
| | | | | attgatgcaactgacaaaggcaaagaatatggc |
| | | | | ggaaaacgcgcatttggtgcgctcggcattggc |
| | | | | ggcttgaaactcaaactgcatcgcgcatgtatt |
| | | | | gcaaaactgtttgagtcttctgaaggtgtattt |
| | | | | gatgcagaggagatttataaactggcaaaagaa |
| | | | | atggcatga |
| Escherichia coli | gcvH | glycine | P0A6T9 | atgagcaacgtaccagcagaactgaaatacagc |
| | (SEQ ID | cleavage | | aaagaacacgaatggctgcgtaaagaagccgac |
| | NO: 17) | systme | | ggcacttacaccgttggtattaccgaacatgct |
| | | (gcv) H | | caggagctgttaggcgatatggtgtttgttgac |
| | | protein | | ctgccggaagtgggcgcaacggttagcgcgggc |
| | | | | gatgactgcgcggttgccgaatcggtaaaagcg |
| | | | | gcgtcagacatttatgcgccagtaagcggtgaa |
| | | | | atcgtggcggtaaacgacgcactgagcgattcc |
| | | | | ccggaactggtgaacagcgaaccgtatgcaggc |
| | | | | ggctggatctttaaaatcaaagccagcgatgaa |
| | | | | agcgaactggaatcactgctggatgcgaccgca |
| | | | | tacgaagcattgttagaagacgagtaa |
| | gcvL | gcv L | P0A9P0 | atgagtactgaaatcaaaactcaggtcgtggta |
| | (SEQ ID | protein | | cttggggcaggccccgcaggttactccgctgcc |
| | NO: 18) | | | ttccgttgcgctgatttaggtctggaaaccgta |
| | | | | atcgtagaacgttacaacacccttggcggtgtt |
| | | | | tgcctgaacgtcggctgtatcccttctaaagca |
| | | | | ctgctgcacgtagcaaaagttatcgaagaagcc |
| | | | | aaagcgctggctgaacacggtatcgtcttcggc |
| | | | | gaaccgaaaaccgatatcgacaagattcgtacc |
| | | | | tggaaagagaaagtgatcaatcagctgaccggt |
| | | | | ggtctggctggtatggcgaaaggccgcaaagtc |
| | | | | actgacgcgctggaactgaaagaagtaccagaa |
| | | | | aaagtggtcaacggtctgggtaaattcaccggg |
| | | | | gctaacaccctggaagttgaaggtgagaacggc |
| | | | | aaaaccgtgatcaacttcgacaacgcgatcatt |
| | | | | gcagcgggttctcgcccgatccaactgccgttt |
| | | | | attccgcatgaagatccgcgtatctgggactcc |
| | | | | cgcctgctggtaatgggtggcggtatcatcggt |
| | | | | ctggaaatgggcaccgtttaccacgcgctgggt |
| | | | | tcacagattgacgtggttgaaatgttcgaccag |
| | | | | gttatcccggcagctgacaaagacatcgttaaa |
| | | | | gtcttcaccaagcgtatcagcaagaaattcaac |
| | | | | ctgatgctggaaaccaaagttaccgccgttgaa |
| | | | | gcgaaagaagacggcatttatgtgacgatggaa |
| | | | | ggcaaaaaagcacccgctgaaccgcagcgttac |
| | | | | gacgccgtgctggtagcgattggtcgtgtgccg |
| | | | | aacggtaaaaacctcgacgcaggcaaagcaggc |
| | | | | gtggaagttgacgaccgtggtttcatccgcgtt |
| | | | | gacaaacagctgcgtaccaacgtaccgcacatc |
| | | | | tttgctatcggcgatatcgtcggtcaaccgatg |
| | | | | ctggcacacaaaggtgttcacgaaggtcacgtt |
| | | | | gccgctgaagttatcgccggtaagaaacactac |
| | | | | ttcgatccgaaagttatcccgtccatcgcctat |
| | | | | accgaaccagaagttgcatgggtgggtctgact |
| | | | | gagaaagaagcgaaagagaaaggcatcagctat |
| | | | | gaaaccgccaccttcccgtgggctgcttctggt |
| | | | | cgtgctatcgcttccgactgcgcagacggtatg |
| | | | | accaagctgattttcgacaaagaatctcaccgt |
| | | | | gtgatcggtggtgcgattgtcggtactaacggc |
| | | | | ggcgagctgctgggtgaaatcggcctggcaatc |
| | | | | gaaatgggttgtgatgctgaagacatcgcactg |
| | | | | accatccacgcgcacccgactctgcacgagtct |
| | | | | gtgggcctggcggcagaagtgttcgaaggtagc |
| | | | | attaccgacctgccgaacccgaaagcgaagaag |
| | | | | aagtaa |
| | gcvP | gcvP | P33195 | atgacacagacgttaagccagcttgaaaacagc |
| | (SEQ ID | protein | | ggcgcttttattgaacgccatatcggaccggac |
| | NO: 19) | | | gccgcgcaacagcaagaaatgctgaatgccgtt |
| | | | | ggtgcacaatcgttaaacgcgctgaccggccag |
| | | | | attgtgccgaaagatattcaacttgcgacacca |
| | | | | ccgcaggttggcgcaccggcgaccgaatacgcc |
| | | | | gcactggcagaactcaaggctattgccagtcgc |
| | | | | aataaacgcttcacgtcttacatcggcatgggt |
| | | | | tacaccgccgtgcagctaccgccggttatcctg |
| | | | | cgtaacatgctggaaaatccgggctggtatacc |
| | | | | gcgtacactccgtatcaacctgaagtctcccag |
| | | | | ggccgccttgaagcactgctcaacttccagcag |
| | | | | gtaacgctggatttgactggactggatatggcc |
| | | | | tctgcttctcttctggacgaggccaccgctgcc |
| | | | | gccgaagcaatggcgatggcgaaacgcgtcagc |
| | | | | aaactgaaaaatgccaaccgcttcttcgtggct |
| | | | | tccgatgtgcatccgcaaacgctggatgtggtc |
| | | | | cgtactcgtgccgaaacctttggttttgaagtg |
| | | | | attgtcgatgacgcgcaaaaagtgctcgaccat |
| | | | | caggacgtcttcggcgtgctgttacagcaggta |
| | | | | ggcactaccggtgaaattcacgactacactgcg |
| | | | | cttattagcgaactgaaatcacgcaaaattgtg |
| | | | | gtcagcgttgccgccgatattatggcgctggtg |
| | | | | ctgttaactgcgccgggtaaacagggcgcggat |
| | | | | attgtttttggttcggcgcaacgcttcggcgtg |
| | | | | ccgatgggctacggtggcccacacgcggcattc |
| | | | | tttgcggcgaaagatgaatacaaacgctcaatg |
| | | | | ccgggccgtattatcggtgtatcgaaagatgca |
| | | | | gctggcaataccgcgctgcgcatggcgatgcag |
| | | | | actcgcgagcaacatatccgccgtgagaaagcg |
| | | | | aactccaacatttgtacttcccaggtactgctg |
| | | | | gcaaacatcgccagcctgtatgccgtttatcac |
| | | | | ggcccggttggcctgaaacgtatcgctaaccgc |
| | | | | attcaccgtctgaccgatatcctggcggcgggc |
| | | | | ctgcaacaaaaaggtctgaaactgcgccatgcg |
| | | | | cactatttcgacaccttgtgtgtggaagtggcc |
| | | | | gacaaagcgggcgtactgacgcgtgccgaagcg |
| | | | | gctgaaatcaacctgcgtagcgatattctgaac |
| | | | | gcggttgggatcacccttgatgaaacaaccacg |
| | | | | cgtgaaaacgtaatgcagcttttcaacgtgctg |
| | | | | ctgggcgataaccacggcctggacatcgacacg |
| | | | | ctggacaaagacgtggctcacgacagccgctct |
| | | | | atccagcctgcgatgctgcgcgacgacgaaatc |
| | | | | ctcacccatccggtgtttaatcgctaccacagc |
| | | | | gaaaccgaaatgatgcgctatatgcactcgctg |
| | | | | gagcgtaaagatctggcgctgaatcaggcgatg |
| | | | | atcccgctgggttcctgcaccatgaaactgaac |
| | | | | gccgccgccgagatgatcccaatcacctggccg |
| | | | | gaatttgccgaactgcacccgttctgcccgccg |
| | | | | gagcaggccgaaggttatcagcagatgattgcg |
| | | | | cagctggctgactggctggtgaaactgaccggt |
| | | | | tacgacgccgtttgtatgcagccgaactctggc |
| | | | | gcacagggcgaatacgcgggcctgctggcgatt |
| | | | | cgtcattatcatgaaagccgcaacgaagggcat |
| | | | | cgcgatatctgcctgatcccggcttctgcgcac |
| | | | | ggaactaaccccgcttctgcacatatggcagga |
| | | | | atgcaggtggtggttgtggcgtgtgataaaaac |
| | | | | ggcaacatcgatctgactgatctgcgcgcgaaa |
| | | | | gcggaacaggcgggcgataacctctcctgtatc |
| | | | | atggtgacttatccttctacccacggcgtgtat |
| | | | | gaagaaacgatccgtgaagtgtgtgaagtcgtg |
| | | | | catcagttcggcggtcaggtttaccttgatggc |
| | | | | gcgaacatgaacgcccaggttggcatcacctcg |
| | | | | ccgggctttattggtgcggacgtttcacacctt |
| | | | | aacctacataaaactttctgcattccgcacggc |
| | | | | ggtggtggtccgggtatgggaccgatcggcgtg |
| | | | | aaagcgcatttggcaccgtttgtaccgggtcat |
| | | | | agcgtggtgcaaatcgaaggcatgttaacccgt |
| | | | | cagggcgcggtttctgcggcaccgttcggtagc |
| | | | | gcctctatcctgccaatcagctggatgtacatc |
| | | | | cgcatgatgggcgcagaagggctgaaaaaagca |
| | | | | agccaggtggcaatcctcaacgccaactatatt |
| | | | | gccagccgcctgcaggatgccttcccggtgctg |
| | | | | tataccggtcgcgacggtcgcgtggcgcacgaa |
| | | | | tgtattctcgatattcgcccgctgaaagaagaa |
| | | | | accggcatcagcgagctggatattgccaagcgc |
| | | | | ctgatcgactacggtttccacgcgccgacgatg |
| | | | | tcgttcccggtggcgggtacgctgatggttgaa |
| | | | | ccgactgaatctgaaagcaaagtggaactggat |
| | | | | cgctttatcgacgcgatgctggctatccgcgca |
| | | | | gaaattgaccaggtgaaagccggtgtctggccg |
| | | | | ctggaagataacccgctggtgaacgcgccgcac |
| | | | | attcagagcgaactggtcgccgagtgggcgcat |
| | | | | ccgtacagccgtgaagttgcggtattcccggca |
| | | | | ggtgtggcagacaaatactggccgacagtgaaa |
| | | | | cgtctggatgatgtttacggcgaccgtaacctg |
| | | | | ttctgctcctgcgtaccgattagcgaataccag |
| | | | | taa |
| Escherichia coli | gcvT | glycine | P27248 | atggcacaacagactcctttgtacgaacaacac |
| | (SEQ ID | cleavage | | acgctttgcggcgctcgcatggtggatttccac |
| | NO: 20) | system T | | ggctggatgatgccgctgcattacggttcgcaa |
| | | protein | | atcgacgaacatcatgcggtacgtaccgatgcc |
| | | | | ggaatgtttgatgtgtcacatatgaccatcgtc |
| | | | | gatcttcgcggcagccgcacccgggagtttctg |
| | | | | cgttatctgctggcgaacgatgtggcgaagctc |
| | | | | accaaaagcggcaaagccctttactcggggatg |
| | | | | ttgaatgcctctggcggtgtgatagatgacctc |
| | | | | atcgtctactactttactgaagatttcttccgc |
| | | | | ctcgttgttaactccgccacccgcgaaaaagac |
| | | | | ctctcctggattacccaacacgctgaacctttc |
| | | | | ggcatcgaaattaccgttcgtgatgacctttcc |
| | | | | atgattgccgtgcaagggccgaatgcgcaggca |
| | | | | aaagctgccacactgtttaatgacgcccagcgt |
| | | | | caggcggtggaagggatgaaaccgttctttggc |
| | | | | gtgcaggcgggcgatctgtttattgccaccact |
| | | | | ggttataccggtgaagcgggctatgaaattgcg |
| | | | | ctgcccaatgaaaaagcggccgatttctggcgt |
| | | | | gcgctggtggaagcgggtgttaagccatgtggc |
| | | | | ttgggcgcgcgtgacacgctgcgtctggaagcg |
| | | | | ggcatgaatctttatggtcaggagatggacgaa |
| | | | | accatctctcctttagccgccaacatgggctgg |
| | | | | accatcgcctgggaaccggcagatcgtgacttt |
| | | | | atcggtcgtgaagccctggaagtgcagcgtgag |
| | | | | catggtacagaaaaactggttggtctggtgatg |
| | | | | accgaaaaaggcgtgctgcgtaatgaactgccg |
| | | | | gtacgctttaccgatgcgcagggcaaccagcat |
| | | | | gaaggcattatcaccagcggtactttctccccg |
| | | | | acgctgggttacagcattgcgctggcgcgcgtg |
| | | | | ccggaaggtattggcgaaacggcgattgtgcaa |
| | | | | attcgcaaccgtgaaatgccggttaaagtgaca |
| | | | | aaacctgtttttgtgcgtaacggcaaagccgtc |
| | | | | gcgtaa |
| | lplA | lipoate- | P32099 | atgtccacattacgcctgctcatctctgactct |
| | (SEQ ID | protein | | tacgacccgtggtttaacctggcggtggaagag |
| | NO: 21) | ligase | | tgtatttttcgccaaatgcccgccacgcagcgc |
| | | | | gttctgtttctctggcgcaatgccgacacggta |
| | | | | gtaattggtcgcgcgcagaacccgtggaaagag |
| | | | | tgtaatacccggcggatggaagaagataacgtc |
| | | | | cgcctggcgcgacgcagtagcggtggcggtgca |
| | | | | gtgttccacgatctcggcaatacctgctttacc |
| | | | | tttatggctggcaagccggagtacgataaaact |
| | | | | atctccacgtcgattgtgctcaatgcgctgaac |
| | | | | gcgctcggcgtcagcgccgaagcgtccggacgt |
| | | | | aacgatctggtggtgaaaaccgtcgaaggcgac |
| | | | | cgcaaagtctcaggctcggcctatcgcgaaacc |
| | | | | aaagatcgcggcttccaccacggcaccttgcta |
| | | | | ctcaatgccgacctcagccgcctggcaaactat |
| | | | | ctcaatccggataaaaagaaactggcggcgaaa |
| | | | | ggcattacgtcggtacgttcccgcgtgaccaac |
| | | | | ctcaccgagctgttgccggggatcacccatgag |
| | | | | caggtttgcgaggccataaccgaggcctttttc |
| | | | | gcccattatggcgagcgcgtggaagcggaaatc |
| | | | | atctccccgaacaaaacgccagacttgccaaac |
| | | | | ttcgccgaaacctttgcccgccagagtagctgg |
| | | | | gaatggaacttcggtcaggctccggcattctcg |
| | | | | catctgctggatgaacgctttacctggggcggc |
| | | | | gtggaactgcatttcgacgttgaaaaaggccat |
| | | | | atcacccgcgcacaggtgtttaccgacagcctc |
| | | | | aacccagcgccgctggaagccctcgccggacga |
| | | | | ctgcaaggctgcctgtaccgcgcagatatgctg |
| | | | | caacaggagtgcgaagcgctgttggttgacttc |
| | | | | ccggaacaggaaaaagagctacgggagttatcg |
| | | | | gcatggatggcgggggctgtaaggtag |
| Escherichia coli | shmt | serine | P0A825 | atgttaaagcgtgaaatgaacattgccgattat |
| | (SEQ ID | hydroxymethyl | | gatgccgaactgtggcaggctatggagcaggaa |
| | NO: 22) | transferase | | aaagtacgtcaggaagagcacatcgaactgatc |
| | | | | gcctccgaaaactacaccagcccgcgcgtaatg |
| | | | | caggcgcagggttctcagctgaccaacaaatat |
| | | | | gctgaaggttatccgggcaaacgctactacggc |
| | | | | ggttgcgagtatgttgatatcgttgaacaactg |
| | | | | gcgatcgatcgtgcgaaagaactgttcggcgct |
| | | | | gactacgctaacgtccagccgcactccggctcc |
| | | | | caggctaactttgcggtctacaccgcgctgctg |
| | | | | gaaccaggtgataccgttctgggtatgaacctg |
| | | | | gcgcatggcggtcacctgactcacggttctccg |
| | | | | gttaacttctccggtaaactgtacaacatcgtt |
| | | | | ccttacggtatcgatgctaccggtcatatcgac |
| | | | | tacgccgatctggaaaaacaagccaaagaacac |
| | | | | aagccgaaaatgattatcggtggtttctctgca |
| | | | | tattccggcgtggtggactgggcgaaaatgcgt |
| | | | | gaaatcgctgacagcatcggtgcttacctgttc |
| | | | | gttgatatggcgcacgttgcgggcctggttgct |
| | | | | gctggcgtctacccgaacccggttcctcatgct |
| | | | | cacgttgttactaccaccactcacaaaaccctg |
| | | | | gcgggtccgcgcggcggcctgatcctggcgaaa |
| | | | | ggtggtagcgaagagctgtacaaaaaactgaac |
| | | | | tctgccgttttccctggtggtcagggcggtccg |
| | | | | ttgatgcacgtaatcgccggtaaagcggttgct |
| | | | | ctgaaagaagcgatggagcctgagttcaaaact |
| | | | | taccagcagcaggtcgctaaaaacgctaaagcg |
| | | | | atggtagaagtgttcctcgagcgcggctacaaa |
| | | | | gtggtttccggcggcactgataaccacctgttc |
| | | | | ctggttgatctggttgataaaaacctgaccggt |
| | | | | aaagaagcagacgccgctctgggccgtgctaac |
| | | | | atcaccgtcaacaaaaacagcgtaccgaacgat |
| | | | | ccgaagagcccgtttgtgacctccggtattcgt |
| | | | | gtaggtactccggcgattacccgtcgcggcttt |
| | | | | aaagaagccgaagcgaaagaactggctggctgg |
| | | | | atgtgtgacgtgctggacagcatcaatgatgaa |
| | | | | gccgttatcgagcgcatcaaaggtaaagttctc |
| | | | | gacatctgcgcacgttacccggtttacgcataa |
| Pseudomonas | ptdh* | phosphonate | 17X- | atgctgccgaaactcgttataactcaccgagta |
| stutzeri | (SEQ ID | dehydrogenase | PTDH- | cacgaagagatcctgcaactgctggcgccacat |
| | NO: 23) | mutant | O69054^{a} | tgcgagctgataaccaaccagaccgacagcacg |
| | | | | ctgacgcgcgaggaaattctgcgccgctgtcgc |
| | | | | gatgctcaggcgatgatggcgttcatgcccgat |
| | | | | cgggtcgatgcagactttcttcaagcctgccct |
| | | | | gagctgcgtgtaatcggctgcgcgctcaagggc |
| | | | | ttcgacaatttcgatgtggacgcctgtactgcc |
| | | | | cgcggggtctggctgaccttcgtgcctgatctg |
| | | | | ttgacggtcccgactgccgagctggcgatcgga |
| | | | | ctggcggtggggctggggaggcatctgagggca |
| | | | | gcagatgcgttcgtccgctctggcaagttccgg |
| | | | | ggctggcaaccacggttctacggcacggggctg |
| | | | | gataacgctacggtcggcttccttggcatgggc |
| | | | | gccatcggactggccatggctgatcgcttgcag |
| | | | | ggatggggcgcgaccctgcagtaccacgcggcg |
| | | | | aaggctctggatacacaaaccgagcaacggctc |
| | | | | ggcctgcgccaggtggcgtgcagcgaactcttc |
| | | | | gccagctcggacttcatcctgctggcgcttccc |
| | | | | ttgaatgccgataccctgcatctggtcaacgcc |
| | | | | gagctgcttgccctcgtacggccgggcgctctg |
| | | | | cttgtaaacccctgtcgtggctcggtagtggat |
| | | | | gaagccgccgtgctcgcggcgcttgagcgaggc |
| | | | | cagctaggagggtatgcggcggatgtattcgaa |
| | | | | atggaagactgggctcgcgcggacaggccacag |
| | | | | cagatcgatcctgcgctgctcgcgcatccgaat |
| | | | | acgctgttcactccgcacatagggtcggcagtg |
| | | | | cgcgcggtgcgactggagattgaacgttgtgca |
| | | | | gcgcagaacatcctccaggcattggcaggtgag |
| | | | | cgcccaatcaacgctgtgaaccgtctgcccaag |
| | | | | gccgagcctgccgcatgttga |
| Arabidopsis | fdh | formate | A0A1P8B9N1 | atggcaatgcgtcaggcagcaaaagcaaccatt |
| thaliana | (SEQ ID | dehydrogenase | (optimized) | cgtgcatgtagcagcagcagctcaagcggttat |
| | NO: 24) | | | tttgcacgtcgtcagtttaatgcaagcagcggt |
| | | | | gatagcaaaaagattgttggtgttttctacaag |
| | | | | gccaacgaatacgcaaccaaaaatccgaatttt |
| | | | | ctgggttgtgttgaaaatgcactgggtattcgt |
| | | | | gattggctggaaagccagggtcatcagtatatt |
| | | | | gttaccgatgataaagaaggtccggattgcgaa |
| | | | | ctggaaaaacatattccggatctgcatgttctg |
| | | | | attagcaccccgtttcatccggcatatgtgacc |
| | | | | gcagaacgtattaagaaagccaaaaatctgaaa |
| | | | | ctgctgctgaccgcaggtattggtagcgatcat |
| | | | | attgatctgcaggcagcagccgcagcaggtctg |
| | | | | accgttgccgaagttaccggtagcaatgttgtt |
| | | | | agcgttgcggaagatgaactgatgcgtattctg |
| | | | | attctgatgcgcaattttgttccgggttataat |
| | | | | caggttgttaaaggcgaatggaatgttgccggt |
| | | | | attgcatatcgtgcatatgatctggaaggtaaa |
| | | | | accattggcaccgttggtgcaggtcgtattggt |
| | | | | aaactgctgttacagcgtctgaaaccgtttggt |
| | | | | tgtaatctgctgtatcatgatcgtctgcagatg |
| | | | | gcaccggaattagaaaaagaaaccggtgccaaa |
| | | | | tttgtcgaagatctgaatgaaatgctgccgaaa |
| | | | | tgtgatgtgattgttattaacatgccgctgacc |
| | | | | gagaaaacccgtggcatgtttaacaaagaactg |
| | | | | attggcaaactgaaaaagggtgtgctgattgtt |
| | | | | aataatgcacgtggtgcaattatggaacgtcag |
| | | | | gccgttgttgatgcagttgaaagcggtcatatt |
| | | | | ggttga |
| | fdh* | formate | fdh: D227Q/ | atgcgtcaggcagcaaaagcaaccattcgtgca |
| | (SEQ | dehydrogenase | L229H | tgtagcagcagcagctcaagcggttattttgca |
| | ID | mutant | (optimized) | cgtcgtcagtttaatgcaagcagcggtgatagc |
| | NO: 25) | | | aaaaagattgttggtgttttctacaaggccaac |
| | | | | gaatacgcaaccaaaaatccgaattttctgggt |
| | | | | tgtgttgaaaatgcactgggtattcgtgattgg |
| | | | | ctggaaagccagggtcatcagtatattgttacc |
| | | | | gatgataaagaaggtccggattgcgaactggaa |
| | | | | aaacatattccggatctgcatgttctgattagc |
| | | | | accccgtttcatccggcatatgtgaccgcagaa |
| | | | | cgtattaagaaagccaaaaatctgaaactgctg |
| | | | | ctgaccgcaggtattggtagcgatcatattgat |
| | | | | ctgcaggcagcagccgcagcaggtctgaccgtt |
| | | | | gccgaagttaccggtagcaatgttgttagcgtt |
| | | | | gcggaagatgaactgatgcgtattctgattctg |
| | | | | atgcgcaattttgttccgggttataatcaggtt |
| | | | | gttaaaggcgaatggaatgttgccggtattgca |
| | | | | tatcgtgcatatgatctggaaggtaaaaccatt |
| | | | | ggcaccgttggtgcaggtcgtattggtaaactg |
| | | | | ctgttacagcgtctgaaaccgtttggttgtaat |
| | | | | ctgctgtatcatcagcgtcatcagatggcaccg |
| | | | | gaattagaaaaagaaaccggtgccaaatttgtc |
| | | | | gaagatctgaatgaaatgctgccgaaatgtgat |
| | | | | gtgattgttattaacatgccgctgaccgagaaa |
| | | | | acccgtggcatgtttaacaaagaactgattggc |
| | | | | aaactgaaaaagggtgtgctgattgttaataat |
| | | | | gcacgtggtgcaattatggaacgtcaggccgtt |
| | | | | gttgatgcagttgaaagcggtcatattggttga |
| Candida boidinii | fdh | | O13437 | atgaagatcgtcttagtcttatacgacgccggc |
| | (SEQ ID | | | aagcacgccgccgatgaagagaagttatacggt |
| | NO: 26) | | | tgcactgaaaacaagttaggtatcgccaactgg |
| | | | | ttaaaggatcaaggccatgaattaatcaccacc |
| | | | | tccgacaaggaaggcggaaactccgtcttggac |
| | | | | caacatatcccagatgccgatatcatcatcaca |
| | | | | actcctttccatcctgcgtacattaccaaggaa |
| | | | | agaatcgacaaggccaagaagttgaaattagtc |
| | | | | gtcgtcgccggcgtgggttccgaccacatcgac |
| | | | | ttggactacatcaaccaaaccggcaagaagatc |
| | | | | tccgtcttggaagtcaccggctccaacgttgtc |
| | | | | tccgtcgccgaacacgtcctcatgaccatgctt |
| | | | | gtcttggtcagaaactttgtcccagcccatgaa |
| | | | | caaatcatcaaccacgactgggaagtcgccgcc |
| | | | | accatcgccaccatcggtgccggtagaatcggt |
| | | | | agaagggtcgaaaacatcgaagaattagtcgcc |
| | | | | tacagagtcttggaaagattagtcccattcaac |
| | | | | ttaccaaaggacgcagaagaaaaggtcggtgcc |
| | | | | attgcaaaggatgcctacgacatcgaaggtaag |
| | | | | ccaaaggaattattatactacgattaccaagcc |
| | | | | caagccgacatcgtcaccatcaacgccccatta |
| | | | | cacgccggtaccaagggtttaatcaacaaggaa |
| | | | | ttattgtctaagttcaagaagggtgcctggtta |
| | | | | gtcaacaccgccagaggtgccatctgtgtcgcg |
| | | | | gaggacgtcgccgccgccctggaatccggtcaa |
| | | | | ttaagaggttacggtggtgacgtctggttccca |
| | | | | caacctgccccaaaggaccatccttggagagac |
| | | | | atgagaaacaaatacggcgccggcaacgccatg |
| | | | | acccctcattactccggtaccaccctggacgcc |
| | | | | caaaccagatacgccgaaggtaccaagaacatc |
| | | | | ttagagtccttcttcaccggtaagtttgactac |
| | | | | agaccacaagacatcatcttattaaacggcgaa |
| | | | | tacatcaccaaggcctatggcaagcacgacaag |
| | | | | aagtga |

^{a}Howe and Van Der Donk, “Temperature-Independent Kinetic Isotope Effects as Evidence for a
Marcus-like Model of Hydride Tunneling in Phosphite Dehydrogenase,” Biochemistry, 58(41):
4260-4268 (2019).

All genes were synthesized with 30 bp overlaps to p70a(2)-deGFP42 to allow Gibson cloning between NdeI/XhoI. The single-plasmid version of Module 1 (Mod1) harbored M. extorquens ftl, fch and mtdA as an operon between the cut sites NdeI/XhoI. E. coli gcvH and lplA were also synthesized with 30 bp overlaps to T3-deGFP and pT7-deGFP to allow Gibson cloning between NcoI/XhoI. His6-tagged versions of Module 2 genes (gcvHLPT and lplA) were also synthesized with a 30 bp overlap to either p70a(2)-deGFP, pT3-deGFP, pT7-deGFP and cloned into those vectors using a similar strategy. Clones were confirmed via DNA sequencing. Plasmids generated for this work can be found in Table 8.

TABLE 8
Table of plasmids

| Strain number | Plasmid name | Description | Source |
|---|---|---|---|
| PPY2510 | pRW10 | p70a(2)-degfp | Garamella et al. ¹ |
| PPY2526 | T3-GFP | pT3-deGFP | Arbor Biosciences |
| PPY2525 | T7-GFP | pT7-deGFP | Arbor biosciences |
| PPY2528 | pRW12 | p70-T3rnap | Arbor biosciences |
| PPY2529 | pRW13 | p70-T7rnap | Arbor biosciences |
| PPY2573 | pRW20 | p70a-M.extorquens_fch | This Study |
| PPY2610 | pSC38 | p70a-M.extorquens_ftl | This Study |
| PPY2611 | pSC39 | p70a-M.extorquens mtdA | This Study |
| PPY2537 | pRW21 | p70a- E. coli_gcvH | This Study |
| PPY2551 | pRW35 | p70a-E.coli_gcvL | This Study |
| PPY2542 | pRW26 | p70a-E.coli_gcvP | This Study |
| PPY2550 | pRW34 | p70a-E.coli_gcvT | This Study |
| PPY2538 | pRW22 | p70a-E.coli_lplA | This Study |
| PPY2535 | pRW19 | p70a-E.coli_shmt | This Study |
| PPY2552 | pRW36 | p70a- M.extorquens | This Study |
| | | ftl_fch_mtdA | |
| PPY2540 | pRW24 | p70a-A.thaliana_fdh* | This Study |
| PPY2541 | pRW25 | P70a-P.stutzeri_ptdh* | This Study |
| PPY2407 | pSC23 | p70a-A.thaliana_fdh | This Study |
| PPY2550 | pRW34 | p70a-E.coli_His6-gcvT | This Study |
| PPY2544 | pRW28 | p70a- E. coli_His6-gcvH | This Study |
| PPY2587 | pKW17 | p70a-E.coli_His6-gcvP | This Study |
| PPY2546 | pRW30 | p70a-E.coli_His6-gcvL | This Study |
| PPY2545 | pRW29 | p70a-E.coli_His6-lplA | This Study |
| PPY2575 | pKW10 | pT3- E. coli_His6-gcvH | This Study |
| PPY2584 | pKW14 | pT7- E. coli_His6-gcvH | This Study |
| PPY2598 | pSC31 | pT3- E. coli_His6-lplA | This Study |
| PPY2602 | pSC35 | pT7- E. coli_His6-lplA | This Study |

^{1.} Garamella et al., “The All E. coli TX-TL Toolbox 2.0: A Platform for Cell-Free Synthetic Biology,” ACS Synth Biol., 5(4):344-55 (2016).

### Linear DNA Formate to Serine Biosynthetic Pathway Construction

The genes ftl, fch, mtdA, ptdh*, gcvHLPT, lplA, shmt were amplified from their respective vectors using primers that bound ˜100 bp upstream from the promoter and downstream the terminator to protect the sequence from exonuclease degradation (Cole and Miklos, “Gene Expression from Linear DNA in Cell-Free Transcription-Translation Systems,” Aberdeen Proving Ground, MD (April 2022)). Specifically, primers RW9/RW10 were used to amplify linear DNA from the p70μ-based plasmids, while GH1/GH2 were used to amplify linear DNA from pT3- and pT7-based plasmids. The T3 and T7 RNA polymerases were amplified from their respective plasmids (p70a-T3 pol, p70a-T7 pol) using primers GH3/GH4, respectively.

Module 1: Synthesis of CH=THF from Formate

Transcription-translation (TXTL) mixture (75% vol.) and 5 nM of each ftl and fch, were added to a PCR tube and brought up to 25 μL using water. Gene expression step: 1 hour at 30° C. shaken at 2.5 g. Biocatalyst dilution step: the reaction was moved to a microcentrifuge tube and diluted to 250 μL, 1 mL, 2.5 mL, and 5 mL using water. Chemical synthesis step: 1 mM of each THF, formate, and ATP were added to the reaction. Chemical synthesis took place over 3 h at 29° C. shaken at 0.0015 g.

Module 1: Synthesis of CH₂-THF from CH=THF

TXTL mixture (75% vol.), 1 mM LiAC, and 5 nM of each mtdA, fdh* were added to a PCR tube and brought up to 25 μL using water. Gene expression step: 16 hours at 30° C. shaken at 2.5 g. Biocatalyst dilution step: the reaction was moved to a microcentrifuge tube and diluted to 250 μL using water. Chemical synthesis step: 1 mM of each CH=THF, formate, and NADPH were added to the reaction, overlayed with argon and sealed. Chemical synthesis took place over 3 h at 29° C. shaken at 0.0015 g.

Module 1: Synthesis of CH₂-THF from Formate

TXTL mixture (75% vol.) and 5 nM of each ftl, fch, mtdA, fdh* were added to a PCR tube and brought up to 25 μL using water. Gene expression step: 1 hour or 16 hours at 30° C. shaken at 2.5 g. Chemical synthesis step for no dilution reactions: stoichiometric concentrations of reactants and co-factors (1 mM of each THF, ATP, NADPH and 2 mM formate) were added to the reaction, overlayed with argon and sealed. For the 10-fold biocatalyst dilution reaction, the reaction was moved to a microcentrifuge tube and stoichiometric concentrations of reactants and co-factors were added to the reactions, diluted to 250 μL using water, overlayed with argon and sealed. Chemical synthesis took place over 3 h at 29° C. shaken at 0.0015 g.

Module 3: Synthesis of serine from CH₂-THF and glycine. A Labcyte Echo 525 was used to dispense TXTL (75% vol.), 100 μM pyridoxal-5-phosphate (PLP) and 5 nM shmt to a 96-well plate and brought up to 5 μl using water. Gene expression step: 16 h at 30° C. shaken at 2.5 g. Biocatalyst dilution step: the reaction was moved to a PCR tube and diluted to 50 μL using water. Chemical synthesis step: 1 mM of each CH₂-THF and glycine were added to the reaction. Chemical synthesis took place over 4 h at 29° C. shaken at 0.0015 g.

Module 1+3+Fdh*/Ptdh*: Synthesis of Serine from Formate and Glycine

A Labcyte Echo 525 was used to dispense 100 μM PLP, and 5 nM of each ftl, fch, and mtdA or the Module 1 operon (Mod1), fdh* or ptdh* and shmt to a 96-well plate. To all DNA mixtures: TXTL (75% vol.) was added by hand and the mixture was brought up to 5 μl using water. Gene expression step: 16 h at 30° C. shaken at 2.5 g. Biocatalyst dilution step: the reaction was moved to a PCR tube and diluted to diluted to 50 μL using water. Chemical synthesis step: stoichiometric concentrations of reactants and co-factors (1 mM of each THF, glycine, NADPH, ATP and 2 mM formate) were added to the reaction, overlayed with argon and sealed. Chemical synthesis took place over 4 h at 29° C. shaken at 0.0015 g.

Module 2+3+Ptdh*: Synthesis of Serine and Glycine from CH₂-THF, Ammonia and Bicarbonate

A Labcyte Echo 525 was used to dispense 100 μM PLP, gcvH, gcvL, gcvP, gcvT, lplA, shmt, and ptdh* to a 96-well plate. TXTL (75% vol.), 100 μM α-lipoic acid were added by hand and the mixture was brought up to 5 μl using water. For non-optimized Module 2 DNA ratio: 40 nM of gcvH and 5 nM of each gcvL, gcvH, gcvP, gcvT, lplA, shmt, and ptdh* were added. For optimized Module 2 linear DNA ratios: 192 nM gcvH (expressed form PT70 or PT3), 1 nM of gcvP, 2 nM gcvL, 2 nM lplA, 4 nM gcvT, and 3 nM each of ptdh*, shmt were added. For the reaction expressing PT3-gcvH, 3 nM of linear pT70-T3RNA was also added. Gene expression step: 16 h at 30° C. shaken at 2.5 g, followed by 2 h at 15° C. shaken at 1.5 g. Biocatalyst dilution step: the reaction was moved to a PCR tube and diluted to 50 μL using 0.1 M Tris HCL pH 8. Chemical synthesis step: To all reactions 20 mM DTT, 100 μM α-lipoic acid and 3 mM H₂NaO₄P were added. For stoichiometric reactions: 2 mM of CH2THF and 1 mM of each NH₃, NaHCO₃, NADH were added. For excess reactions: 10 mM of each NH₃ and NaHCO₃ were added while the concentrations of all other reagents and cofactors were held constant. The reaction was overlayed with argon and sealed. Chemical synthesis took place over 4 h at 29° C. shaken at 0.0015 g.

P. stutzeri Phosphonate Dehydrogenase Substrate Preference

TXTL mixture (75% vol.), 5 nM of ptdh* was added to a PCR tube and brought up to 25 μL using water. Gene expression step: 16 hours at 30° C. shaken at 2.5 g. Biocatalyst dilution step: the reaction was moved to a microcentrifuge tube and diluted to 250 μL using water. Chemical synthesis step: either 1 mM of NAD⁺, 1 mM of NADP⁺ or 1 mM of each NAD⁺ and NADP⁺ were added to the reaction. Cofactor regeneration took place over 4 h at 29° C. shaken at 0.0015 g.

Module 1+2+3+Ptdh*: Synthesis of Serine from Formate, Ammonia and Bicarbonate

Labcyte Echo 525 was used to dispense 100 μM PLP, Mod1, mtdA, gcvH, gcvL, gcvP, gcvT, lplA, shmt, and ptdh* to a 96-well plate. For non-optimized Module 2 gene ratios: 40 nM of gcvH and 5 nM of each Mod1, gcvL, gcvH, gcvP, gcvT, lplA, shmt, and ptdh* were added. For optimized Module 2 gene ratios: 3 nM Mod1, 192 nM P_{T3}-gcvH, 1 nM of gcvP, 2 nM gcvL, 2 nM lplA, 4 nM gcvT, 3 nM shmt, 3 nM ptdh*, and 3 nM pT70-T3RNA were added. For 2× mtdA reactions: 3 nM mtdA was added. For 2× shmt reactions: an additional 3 nM shmt were added. To all DNA mixtures, TXTL (75% vol.), 100 μM α-lipoic acid were added by hand and brought up to 5 μl using water. Gene expression step: 16 h at 30° C. shaken at 2.5 g, followed by 2 h at 15° C. shaken at 1.5 g. Biocatalyst dilution step: the reaction was moved to a PCR tube and diluted to 50 μL using 0.1 M Tris HCL pH 8. Chemical synthesis step: 20 mM DTT, 100 μM α-lipoic acid and 3 mM H₂NaO₄P were added. For stoichiometric reactions: 2 mM of each THF, formate, NADPH, ATP, and 1 mM of each NH₃, NaHCO₃, NADH were added. For 10× reactants reactions: 10 mM of each formate, NH₃ and NaHCO₃ was used while keeping concentration of all other components constant. For 10× less THF reactions: 0.2 mM THF concentration was used while keeping concentration of all other components constant. The reaction was overlayed with argon and sealed. Chemical synthesis took place over 4 hours at 29° C. shaken at 0.0015 g.

### Quantification of Protein Levels of Module 2 Enzymes

A Labcyte Echo 525 was used to dispense 100 μM PLP, various concentrations of His-tagged PT70 gcvHLPT and lplA. For P_{T3} and P_{T7} gcvH and lplA reactions, 3 nM P_{T70}-T3RNA or P_{T70}-T7RNA were also added. To all DNA mixtures: TXTL (75% vol.) and 100 μM α-lipoic acid were added by hand and brought up to 5 μl using water. Gene expression step: 16 h at 30° C. shaken at 2.5 g. Western Blot: 2 μL of each reaction were loaded along with NUPAGE LDS sample buffer into each well of a 4-12% Bis-Tris gel and run using an XCell SureLock Mini-Cell Electrophoresis System and NuPAGE MES SDS running buffer. The protein bands were transferred to a nitrocellulose paper using iBlot Dry Blotting System. Proteins were washed between steps with Tris-buffered saline, blocked with a bovine serum albumin buffer, and labeled with a monoclonal anti-polyhistidine antibody (mouse) followed by an anti-mouse IgG-alkaline phosphatase antibody (goat). The blot was developed using a nitro-blue tetrazolium chloride (NBT) and 5-bromo-4-chloro-3′-indolyphosphate p-toluidine salt (BCIP) color developing substrate system.

### Amino Acid Derivatization

For liquid chromatography/mass spectrometry (LC/MS) quantification, serine and glycine were derivatized to their Fmoc protected versions using 9-fluorenylmethoxycarbonyl chloride51. At this point, 1 mM Boc-Serine was added to the reaction mixture for use as an internal standard in the LC/MS quantification of glycine and serine. After stopping the CFE-based biocatalyst with 5% acetic acid in methanol to trigger protein denaturation, the reaction was centrifuged and diluted 10-fold with water. To 25 μl of the diluted sample, 100 μl 3 mM Fmoc-Cl dissolved in acetone was added at a pH 8.3 (with saturated NaHCO₃). Fmoc derivatization of amino acids was done at room temperature for 10 minutes. The Fmoc-derivatized amino acids were extracted using ethyl acetate and the dried sample was resuspended in 200 μl methanol.

### Liquid Chromatography/Mass Spectrometry (LC/MS)-Based Chemical Analysis

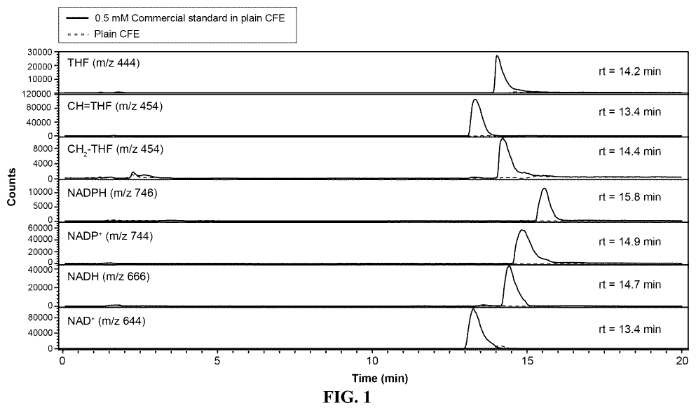

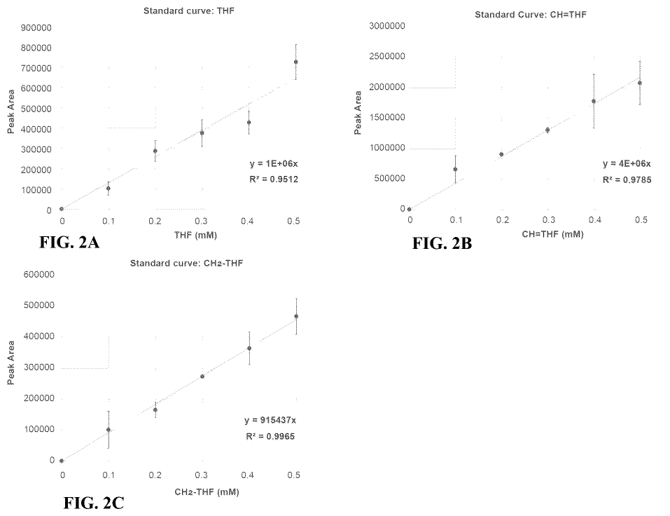

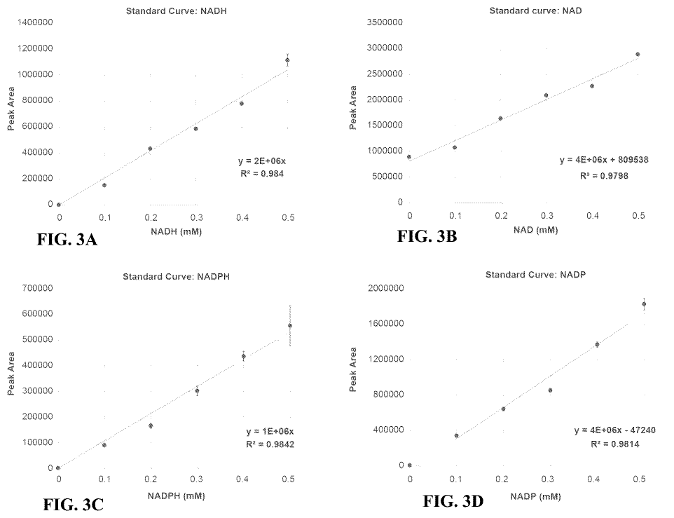

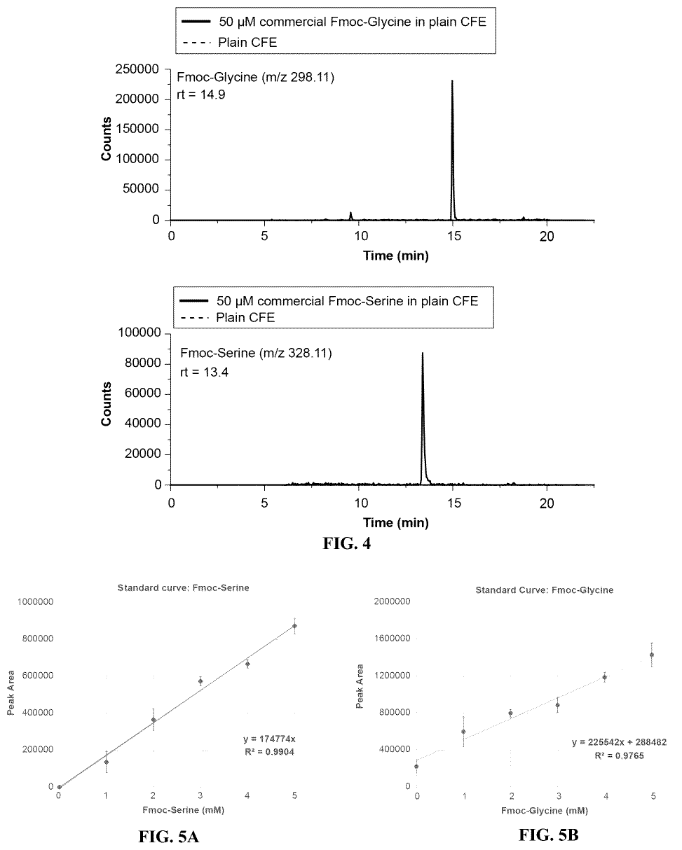

All Module 1 reactions were stopped by adding 5% acetic acid in methanol spiked with 4 mM catechol (internal standard for CH=THD and CH₂-THF quantification) to trigger protein denaturation. The denatured reactions were centrifuged at 16,000 g for 15 min. LC/MS conditions: THF, CH=THF, CH₂-THF, NAD⁺, NADPH, NADP⁺, NADH were quantified using an Agilent 1100/1260 HPLC equipped with an Agilent 6120 Single Quadrupole MS, using a Poroshell 120 SB-C18 3.0 mm×50 mm×2.7 μm column and an electrospray ion source. Column temperature was kept constant at 28° C. The LC method was based on Chen et al.52. LC conditions: Solvent A—water with 3% methanol, 10 mM tributylamine and 15 mM acetic acid, Solvent B—methanol. Gradient: 0 min, 0% B; 2.5 min, 0% B; 5 min, 50% B; 14 min, 95% B; 15 min, 0% B; 20 min, 0% B. MS acquisition: Selective ion monitoring (SIM) in negative ion mode was used to detect and quantify THF (m/z 444), CH=THF (m/z 454), CH₂-THF (m/z 456) (FIG. 1). Positive ion mode was used to detect NADH (m/z 666), NAD⁺ (m/z 664), NADPH (m/z 746), and NADP⁺ (m/z 104) (FIG. 1). Commercial THF, CH=THF, CH₂-THF, NADH, NAD⁺, NADPH and NADP⁺ were used to determine retention times and generate standard curves for chemical quantification (FIGS. 2A-2C and 3A-3D). The Fmoc-derivatized amino acids were quantified using Agilent 1260 Infinity II HPLC system equipped with an Agilent Q-TOF 6530 detector, using Poroshell 120 SB-C18 3.0 mm×50 mm×2.7 μm column. LC conditions: Solvent A—water with 0.1% formic acid, Solvent B—methanol with 0.1% formic acid. Gradient: 0 min, 0% B; 2.5 min, 0% B; 5 min, 50% B; 15 min, 100% B; 15 min, 0% B; 20 min, 0% B. MS acquisition: Extracted ion chromatogram (EIC) in positive ion mode was used to detect and quantify Fmoc-Serine (m/z 328.11), and Fmoc-Glycine (m/z 298.11) (FIG. 4). Fmoc derivatized commercial glycine and serine were used to determine retention times and generate standard curves for chemical quantification (FIGS. 5A-5B).

### Example 2—Formate-to-Serine CFE-Based Biocatalyst Overview

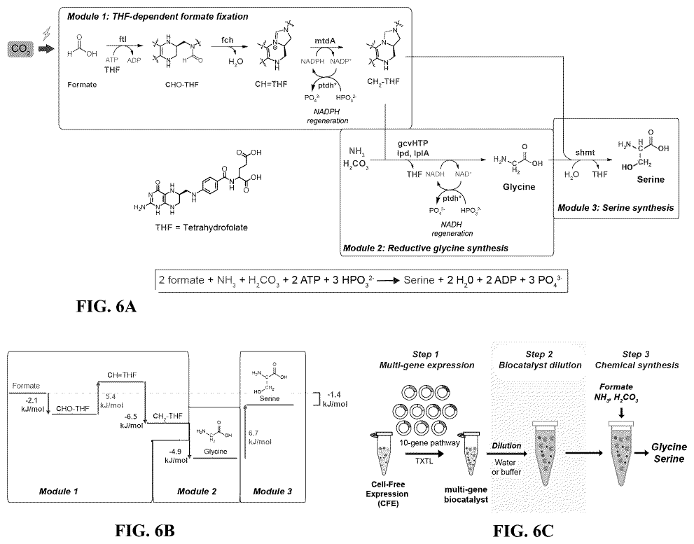

To facilitate multi-enzyme biocatalyst assembly and optimization, the pathway was divided into three modules. Module 1, THF-dependent formate fixation, attaches the C1 from formate to THF to generate the C1 carrier molecule CH₂-THF using 1 ATP and 1 NADPH. Module 2, reductive glycine synthesis, brings together CH₂-THF, bicarbonate (H₂CO₃) and ammonia (NH₃) to synthesize glycine using 1 NADH and recycling THF in the process. Module 3, serine synthesis, incorporates the C1 from a second CH₂-THF onto glycine to synthesize serine and recycle a second THF. Because both formate and bicarbonate can be directly obtained from CO₂, synthesis of glycine captures two C02 equivalents, while serine synthesis captures a total of three CO₂ equivalents per molecule (FIG. 6A).

Thermodynamic analysis of the formate-to-serine biocatalyst revealed it to be marginally thermodynamically favorable at ΔG°′=−1.4 kJ/mol40 (FIG. 6B). As there is no major thermodynamic sink in the system, efficient co-factor (NAD(P)H/THF) regeneration is pivotal to keep cofactor concentrations high and drive carbon flux forward through the pathway based on Chatelier's Principle. While Modules 2 and 3 recycle THF, NAD(P)H regeneration can be conceived as an independent unit of operation. For NAD(P)H regeneration, we first evaluated the use of formate as both the carbon and electron source by using formate dehydrogenase (fdh). As fdh releases one CO₂ per NAD(P)H regenerated, we also evaluate the use of an engineered phosphonate dehydrogenase (ptdh* ((SEQ ID NO: 10)) that uses phosphonate as the reducing power, thus enabling the use of formate only as the carbon source and sustaining a more carbon negative process.

### Example 3—Volumetric Expansion of the CFE-Based Biocatalyst

A major challenge to scale up a CFE-based multi-enzyme biocatalyst for the synthesis of large-volume low-cost chemicals is the high cost of the cell lysate (˜$90/L (Rasor, et al., “Toward Sustainable, Cell-free Biomanufacturing,” Curr Opin Biotech, 69:136-144, (2021)) when compared to microbial-based catalysts. Towards addressing this challenge, we introduced a CFE-based biocatalyst dilution step ahead of the chemical synthesis step to enable greater substrate loading and achieve greater product levels for the same CFE reagent cost (FIG. 6C). Briefly, during the multi-gene expression (Step 1), the transcription-translation conditions optimal in the CFE system (Garamella et al., “The All E-coli TX-TL Toolbox 2.0: A Platform for Cell-Free Synthetic Biology,” ACS Synth Biol, 5:344-355 (2016)) are maintained; specifically, the ratio of the cell lysate, energy molecules and cofactors to buffer. In Step 2, the CFE-based biocatalyst is diluted with water or inexpensive buffer to volumetrically expand the reaction. To initiate the chemical synthesis (Step 3), the substrates and cofactors are added to the reaction. Of note, the biocatalyst is used without purification during the chemical synthesis step. Volumetric expansion of a CFE-based biocatalyst 1) dilutes endogenous CFE reactions, reducing the siphoning of pathway intermediates to other fates, 2) enables greater substrate loading, and 3) if the biocatalyst maintains high conversion efficiency, it achieves higher chemical synthesis levels. Volumetric expansion of the formate to serine biocatalyst is possible because the pathway does not rely on endogenous CFE reactions and regenerates its own cofactors. If volumetric expansion does enable greater chemical synthesis levels, it could significantly reduce bioproduction costs, which is key for the eventual scale up of the CFE-based process as CFE has a higher price point than microbial fermentation (Claassens et al., “A Critical Comparison of Cellular and Cell-free Bioproduction Systems,” Curr Opin Biotech, 60:221-229 (2019)). Importantly, CFE-based biocatalysts could work at a wider range of pHs, solvents, and temperatures than microbial catalysts. Additionally, unlike microbial biocatalysts, CFE-based biocatalysts could enable product formation at maximal velocity as there are no membranes to limit substrate or product diffusivity (Claassens et al., “A Critical Comparison of Cellular and Cell-free Bioproduction Systems,” Curr Opin Biotech, 60:221-229 (2019)).

### Example 4—Module 1: THF-Dependent Formate Fixation

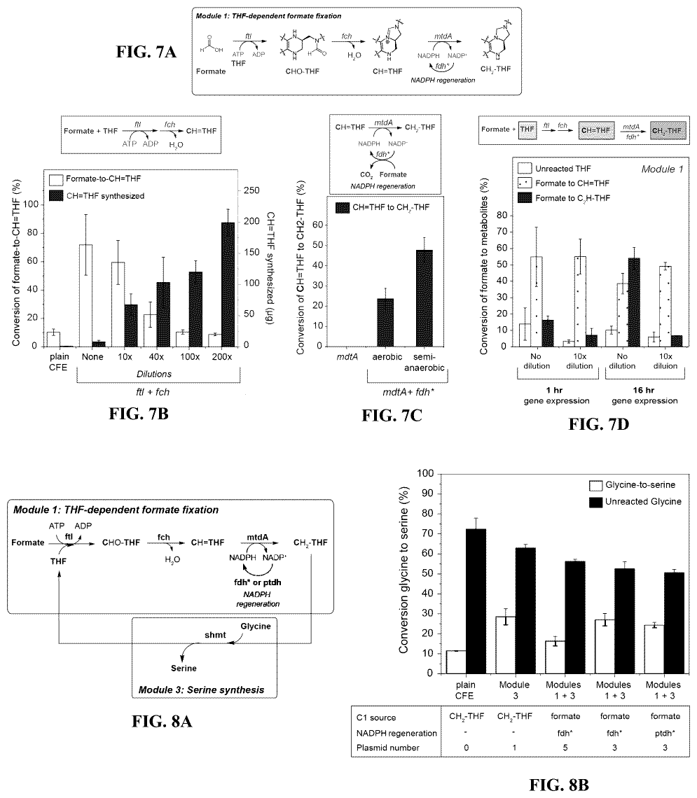

Module 1 leverages Methylobacterium extorquens formate-THF ligase (ftl), methenyl-THF cyclohydrolase (fch) and methylene THF dehydrogenase (mtdA) to fix formate to THF to ultimately generate CH₂-THF (FIG. 7A). Because CHO-THF rapidly cyclizes to CH=THF, ftl and fch were studied as a pair (FIG. 7B). Plain CFE, i.e., CFE without pathway genes but with added substrates and cofactors, resulted in 10% formate conversion to CH=THF due to spontaneous condensation of formate to THF at pH-7 followed by non-enzymatic cyclization to CH=THF. Direct expression of ftl and fch in CFE generates the ftl/fch biocatalyst, which resulted in 72% conversion of formate to CH=THF. Ten-fold volumetric expansion of the ftl/fch CFE-based biocatalyst with water followed by supplementation with the same concentrations of substrate and cofactors at the chemical synthesis step resulted in a slightly lower formate fixation (60%), but achieved an 8-fold increase in total CH=THF synthesis (68 μg). To test the limits of the volumetric expansion strategy, the ftl/fch biocatalyst was diluted 200-fold. Although formate fixation dropped to 9% as the concentration of the biocatalyst decreased, there was a 25-fold improvement in CH=THF synthesis (199 μg). Taken together, decoupling gene expression from chemical synthesis is a viable strategy to increase the chemical levels produced by CFE-based biocatalysts. The enzyme activity was retained during the volumetric expansion of the CFE-based biocatalyst, allowing greater substrate and cofactor loading, thus higher synthesis levels of the desired product.

Next, the NADPH-dependent reduction of CH=THF to CH₂-THF was evaluated (FIG. 7C). Direct expression of mtdA in CFE followed by 10-fold dilution and supplementation with stoichiometric amounts of CH-THF and NADPH did not result in detectable concentrations of CH₂-THF. As CH=THF reduction to CH₂-THF is near thermodynamic equilibrium, in situ NADPH regeneration was introduced to keep NADPH concentration high and drive the reaction forward. Direct expression of mtdA and a mutant of A. thaliana formate dehydrogenase known to recycle NADP+ (fdh*) (Ihara et al., “Light Driven CO₂ Fixation by using Cyanobacterial Photosystem I and NADPH-dependent Formate Dehydrogenase,” PLoS One, 8:e71581 (2013)) in CFE resulted in 23% conversion of CH=THF to CH₂-THF. Thus, efficient NADPH regeneration is pivotal for CH=THF reduction. The oxygen sensitivity of mtdA (Huang et al.; “The Hydride Transfer Process in NADP-dependent Methylene-tetrahydromethanopterin Dehydrogenase,” J Mol Biol, 432:2042-2054 (2020)) led to the evaluation of CH=THF reduction under semi-anaerobic conditions, which resulted in a 48% conversion of CH=THF to CH₂-THF.

Finally, all Module 1 genes (ftl, fch, mtdA) and fdh* were directly expressed in CFE to generate the Module 1 biocatalyst (FIG. 7D). Supplementation of the Module 1 biocatalyst with stoichiometric concentrations of formate, THF, ATP, and NADPH resulted in 16% conversion from formate-to-CH₂-THF. Interestingly, CH=THF accumulates in the system (55%), hinting at mtdA being the rate limiting step. We see a similar formate conversion trend when the biocatalyst is diluted 10-fold. Hypothesizing that the 1-hour direct CFE may be limiting the amount of biocatalyst generated, the gene expression step was increased to 16 hours. The Module 1 biocatalyst now achieved a 54% conversion of formate to CH₂-THF, supporting the idea that the system was biocatalyst limited. Ten-fold dilution of the 16-hour gene expressed Module 1 biocatalyst resulted in only an 8% conversion of formate to CH₂-THF. Close to 50% of formate was caught at CH=THF, confirming the hypothesis that mtdA is the rate-limiting step. We did not optimize Module 1 further as we hypothesized that successful implementation of Modules 2 and 3 that use CH₂-THF as a substrate would pull on CH=THF to be converted to CH₂-THF as needed.

### Example 5—Module 3: Serine Synthesis

Given the success of volumetric expansion, all subsequent chemical synthesis steps were run at a 10-fold biocatalyst dilution. Module 1 terminates in CH₂-THF, which enters both reductive glycine synthesis (Module 2) and serine synthesis (Module 3). Due to the complexity of Module 2, which requires multiple substrates and cofactors (CH₂-THF, NH₃, H₂CO₃, NADH) to form glycine, we first evaluated Module 3, which is composed of a single enzyme, E. coli serine hydroxymethyltransferase (shmt). Module 3 brings together glycine and CH₂-THF to produce serine recycling THF in the process (FIG. 8A). Plain CFE supplemented with CH₂-THF and glycine results in ˜11% conversion to serine due to the endogenous shmt in the CFE. The Module 3 catalyst supplemented with CH₂THF and glycine achieves 29% conversion to serine (FIG. 8B). Next, we assessed if Module 1 could generate sufficient CH₂-THF for Module 3 to drive serine synthesis. A Module 1+3+fdh* biocatalyst supplemented with equimolar concentrations of formate, THF and glycine achieved a 16% conversion of glycine-to-serine. The observed 13% drop when comparing to the glycine-to-serine conversion of Module 1 vs. Module 1+3+fdh* could be attributed to the larger number of plasmids used to generate the Module 1+3+fdh* biocatalyst (5) when compared to Module 3 (1) biocatalyst. To reduce plasmid burden, the Module 1 genes (ftl, fch and mtdA) were cloned as an operon in a single plasmid, while fdh* and shmt were kept in separate plasmids. The 3-plasmid Module 1+3+fdh* biocatalyst improved glycine-to-serine conversion to 27%. Taken together reducing the plasmids burden improved glycine-to-serine conversion 2-fold. Of note, unlike Module 1 intermediates that are orthogonal to the E. coli-based CFE machinery, both glycine and serine can be consumed by background CFE reactions. Thus, the 27% conversion of glycine to serine achieved may be a lower limit of the overall process.

Finally, we increased the carbon negativity of the process by swapping fdh* with a previously engineered Pseudomonas stutzeri phosphonate dehydrogenase (ptdh*) that uses polyphosphonate as the reducing power to regenerate both NADPH and NADH (Howe and Van Der Donk, “Temperature-independent Kinetic Isotope Effects as Evidence for a Marcus-like Model of Hydride Tunneling in Phosphite Dehydrogenase,” Biochemistry, 58(41):4260-4268 (2019), Nguyen and Agarwal, “A Leader-Guided Substrate Tolerant RiPP Brominase Allows Suzuki-Miyaura Cross-Coupling Reactions for Peptides and Proteins,” Biochemistry, 62(12):1838-1843 (2023)). A Module 1+3+ptdh* biocatalyst supplemented with equimolar concentrations of formate, THF and glycine resulted in 24% conversion of glycine-to-serine. Although use of ptdh* results in a slightly lower glycine-to-serine conversion, ptdh* enables 1) the use of formate exclusively as a carbon source, 2) does not release CO₂ release per NAD(P)+ recycled, and 3) enables the use of a single enzyme to recycle both NADPH and NADH. Thus, we used ptdh* in subsequent experiments.

### Example 6—Module 3: Reductive Glycine Synthesis

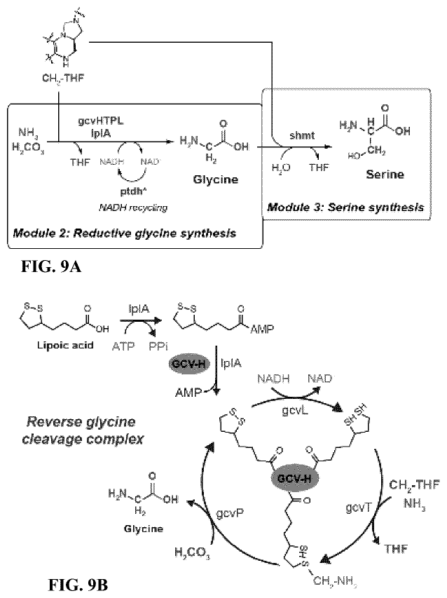

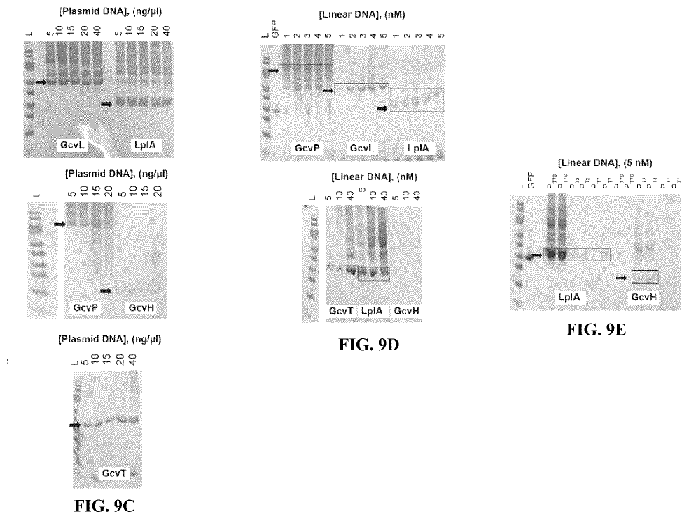

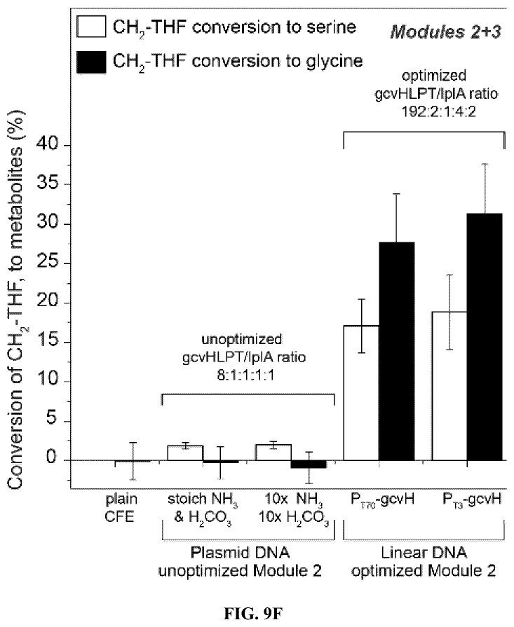

In Module 2, the glycine cleavage complex (gcv) is run in reverse, converting CH₂-THF, H₂CO₃ and NH₃ to glycine using one NADH in the process (FIG. 9A). Specifically, Module 2 is composed of the four gcv genes, gcvH, gcvT, gcvP, and gcvL, and lipoate protein ligase (lplA) that loads lipoic acid onto gcvH to enable its function (FIG. 9B). Although reverse gcv has been implemented in microbes (Bang and Lee, “Assimilation of Formic Acid and CO₂ by Engineered Escherichia coli Equipped with Reconstructed One-carbon Assimilation Pathways,” P Natl Acad Sci USA 115:E9271-E9279 (2018), Bang et al., “Escherichia coli is Engineered to Grow on CO₂ and Formic Acid,” Nat Microbiol., 5(12):1459-1463 (2020)), unique challenges arise when moving this system to CFE. First, CFE lacks the biosynthetic pathways for lipoic acid and pyridoxal phosphate, which need to be supplemented. Second, in CFE, the four gcv genes are expressed from synthetic promoters rather than their endogenous ones. Thus, the gcvHLPT gene ratio to be directly expressed in CFE needs to be identified to achieve the optimal gcvHLPT enzyme ratio of 8:1:1:1 previously determined in purified enzyme systems (Xu et al., “Improvement of Glycine Biosynthesis from One-carbon Compounds and Ammonia Catalyzed by the Glycine Cleavage System In Vitro,” Eng Life Sci 22:40-53 (2022)). As a starting point, we assumed similar transcription-translation levels for all gcvHLPT genes and used plasmid concentrations that reflect an 8:1:1:1 ratio.

The CFE-based Module 2+3+ptdh* biocatalyst supplemented with equimolar concentrations of CH₂-THF, H₂CO₃, NH₃ and NADH resulted in 1.8% conversion of CH₂-THF-to-serine. Use of a 10-molar excess of NH₃ and H₂CO₃ increased conversion slightly to 1.9%. Given the 24% conversion for the Modules 1+3+ptdh* biocatalyst, a 1.8% conversion for the Module 2+3+ptdh* biocatalyst would significantly impair the synthesis of serine from formate. We hypothesized that the four gcv genes (SEQ ID NOS: 17-20) did not have similar transcription-translation levels, thus we set out to determine the relationship between the concentration of Module 2 genes directly expressed in CFE o their protein synthesis levels. As FIG. 9C shows, we found robust expression of gcvL, gcvP and lplA, all peaking at 5 ng/μl, and gcvT peaking at 20ng/μl (see FIG. 11 for complete western blots). Expression of gcvH, however, was markedly lower, and increases in plasmid concentration did not significantly increase protein concentrations. Therefore, gcvH expression limits the Module 2 biocatalyst.

To improve gcvH expression, we took a two-pronged approach: 1) we investigated the use of linear DNA to access greater gene loading into the CFE and 2) we evaluated the use of stronger promoters to drive gcvH expression. The formate-to-serine pathway is a 7-plasmid system. Further increasing the plasmid DNA concentration in the system led to viscosity issues, thus continuing to increase gcvH plasmid concentration was not a viable solution. To address this issue, Module 2 was moved to a linear DNA system for direct gene expression in a CFE optimized to prevent nucleic acid degradation (Sun et al., “Linear DNA for Rapid Prototyping of Synthetic Biological Circuits in an Escherichia coli Based TX-TL Cell-Free System,” ACS Synth Biol, 3:387-397 (2014)). Using the pixel intensity of the Western Blot protein bands, we calculated the approximate protein ratios between gcvP, gcvL and lplA to be 1:3:4 when 2-4 nM of either gcvP, gcvL or lplA was directly expressed in CFE (FIGS. 9D and 12). The expression levels of gcvT and lplA were similar to one another, while the expression of gcvH was very low, even up to 40 mM.

To further improve gcvH expression, we moved gcvH from control by the medium strength promoter PT70 to the stronger promoters P_{T3} and P_{T7}. As shown in FIG. 9E, P_{T3}-gcvH results in significantly higher gcvH levels when compared to P_{T70}-gcvH or P_{T}7-gcvH. Interestingly, use of P_{T3} did not improve the expression of other Module 2 genes. For example, P_{T70}-lplA results in higher protein levels than P_{T3} or P_{T}7-lplA. Taken together, to achieve similar protein concentrations of all Module 2 genes, the molar gcvHLPT/lplA gene ratio should be ˜12:3:1:4:4. To achieve a gcvHLPT protein ratio of 8:1:1:1, the calculated DNA molar ratio of gcvHLPT/lplA should be ˜96:3:1:4:4.

The optimal calculated Module 2 gene ratio (gcvHLPT/lplA=96:3:1:4:4) was obtained by expressing each gene independently in CFE. However, the CFE-based multi-enzyme biocatalyst requires co-expression of all five Module 2 genes simultaneously. Thus, it is possible that CFE capacity, i.e. RNA polymerases, ribosomes, tRNAs and amino acids available for protein synthesis, is reached before the maximum protein concentrations for each Module 2 gene is achieved. Nevertheless, it was assumed that the relative expression of Module 2 genes will remain approximately the same as gene expression is sequence dependent. To ensure sufficient gcvH protein synthesis in a CFE system that may be close to protein expression capacity, we experimentally tested the gcvHLPT/lplA ratio of 192:2:1:4:2. As shown in FIG. 9F, the optimized Module 2 (P_{T70}-gcvH)+3+ptdh* biocatalyst supplemented with stoichiometric concentrations of CH₂-THF, NH₃ and H₂CO₃ resulted in 17% conversion of CH₂-THF-to-serine with an additional 27% conversion of CH₂-THF-to-glycine. The optimized Module 2 (P_{T3}-gcvH)+3+ptdh* biocatalyst improved CH₂-THF-to-serine conversion slightly to 19% with an additional 31% conversion of CH₂-THF-to-glycine. Taken together, the optimized Module 2 catalyst achieves a combined CH₂-THF-to-glycine and serine conversion of 50% when using P_{T3}-gcvH. This is a 33-fold improvement over the combined CH₂-THF-to-serine and glycine conversion of the unoptimized Module 2 catalyst.

### Example 7—Synthesis of Serine and Glycine from Formate, Bicarbonate and Ammonia

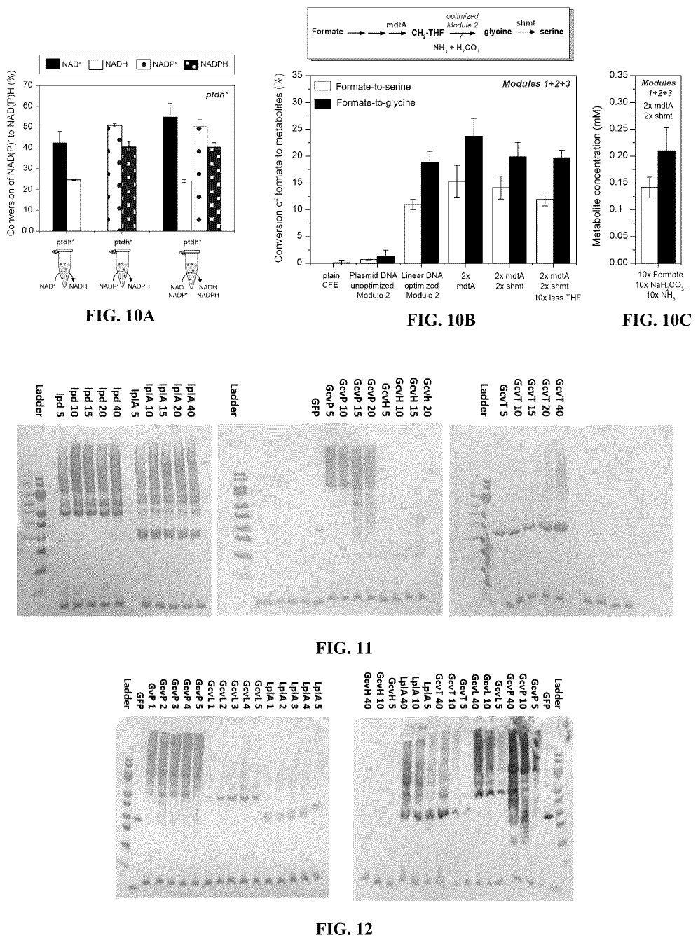

We assembled the formate-to-serine biocatalyst by directly expressing Module 1, Module 2 (gcv lplA, P_{T3}-gcvH), Module 3 and ptdh* in CFE. In this multi-enzyme biocatalyst, ptdh* would regenerate both NADPH (Module 1) and NADH (Module 2). Thus, we first sought to understand any substrate preference by ptdh* through evaluating its ability to regenerate NADPH and NADH either in isolation or in an equimolar mixture. As shown in FIG. 10A, ptdh* regenerated 40% of the NADPH and 23% of NADH both in isolation and in an equimolar mixture of both substrates. With an efficient NAD(P)H regeneration system in hand, we measured the conversion of formate into glycine and serine (FIG. 10B). Using plasmids DNA to express Module 1+2+3 at unoptimized Module 2 resulted in a combined 2% conversion of formate-to-serine and glycine. Using linear DNA to express Module 1+2+3 at optimized Module 2 improved the combined formate-to-serine and glycine to 30%.

### Example 8—Metabolic Optimization of Formate-to-Serine Conversion

To further improve the conversion of formate-to-serine we pursued metabolic “push” and “pull” strategies. First, knowing that mdtA limits CH=THF reduction to CH₂-THF in Module 1 (FIG. 7D), we introduced a 2-fold molar excess of mtdA linear DNA as part of the formate-to-serine CFE-based biocatalyst. This “push” strategy resulted in both improved formate-to-serine (15%) and formate-to-glycine (24%) conversion (FIG. 10B). In terms of biosynthetic productivity, the “push” strategy improved the biosynthetic productivity of the CFE-based biocatalyst from 2.9 to 4.0 mg/L/h with respect to serine and from 3.5 to 4.5 mg/L/h with respect to glycine. Next, to address the buildup of glycine, we introduced a 2-fold molar excess of shmt linear DNA as part of the biocatalyst. This “pull” strategy actually reduced the formate conversion to serine (14%) or glycine (20%). Taken together, using a 2-fold excess of mdtA as part of the formate-to-serine biocatalyst resulted in a combined 39% conversion of formate-to-serine and glycine.

Thus far, stoichiometric concentrations of formate and the key cofactor THF have been used to evaluate the formate-to-serine biocatalyst. To investigate whether formate-to-serine synthesis could be run catalytically, we lowered the THF concentration 10-fold when compared to formate, i.e. 10% cofactor loading. As shown in FIG. 10B, the formate-to-serine biocatalyst efficiently recycles THF resulting in 12% formate-to-serine and 20% formate-to-glycine conversion. Indeed, the combined formate to serine and glycine conversion with 10-fold less THF (32%) is comparable to that obtained when THF was added at stoichiometry (39%). Lower cofactor loading reduces the cost of the CFE-based process, supporting the scale up of CFE-based biocatalyst.

Finally, we examined whether the CFE-based biocatalyst was running at enzyme capacity by adding a 10-fold excess of each formate, ammonia and bicarbonate while keeping the concentration of the co-factors constant at 1 mM (FIG. 10C). We find that, under excess substrate, the biocatalyst achieved a similar total serine and glycine concentration, 0.2 mM and 0.14 mM, respectively as when running the system at 1 mM concentration of reactants. Taken together, at 1 mM concentration, the formate-to-serine biocatalyst is at capacity.

### Example 9—Discussion of Examples 1-8

A 10-enzyme CFE-based biocatalyst for the de novo synthesis of the industrially-relevant amino acids serine and glycine from formate, bicarbonate, and ammonia was successfully engineered. Since CO₂ can be electrochemically converted to formate, the formate-to-serine biocatalyst enables the carbon negative synthesis of glycine and serine capturing 3 CO₂ molecules per serine synthesized. The combined 39% conversion of formate to serine and glycine surpasses the previous formate to glycine conversion (22%) achieved via rGS using purified enzyme systems (Wu et al., “Enzymatic Electrosynthesis of Glycine from CO₂ and NH₃,” Angewandte Chemie, 135:e202218387 (2023)). The system regenerates NAD(P)H and THF well, even capable of converting formate-to-serine and glycine using 10-fold lower concentration of THF and achieving similar conversion rates as when THF is added at stoichiometry. These results support the future use of the CFE-based biocatalyst as part of a continuous chemical synthesis process.

When compared to traditional biocatalysts that require microbial enzyme expression followed by purification before use, CFE-based biocatalysts are more versatile as they can be produced on-demand and in situ via direct expression of DNA in CFE. The ability to rapidly generate CFE-based biocatalysts enabled the rapid screening of different enzyme isoforms, reagent stoichiometries and DNA expression conditions, i.e. plasmid vs. linear DNA. Additionally, the CFE-based biocatalyst can be used without purification. The dilution of the biocatalyst with inexpensive buffer, i.e. volumetric expansion, explored in this work enabled increased substrate loading resulting in overall greater product amounts while reducing the carbon flux diverted to endogenous CFE reactions. Specifically, in this work, for the initial two-step pathway to incorporate the C1 donor group into THF, a 200-fold dilution of the CFE biocatalyst allowed greater substrate loading and yielded 25 times more product than the undiluted reaction with the same amount of enzyme. The further development of these technologies could enable the production of a wide variety of industrial products¹¹ with 100% carbon and energy efficiency.

Two aspects were pivotal in achieving the combined 39% formate-to-serine and glycine conversion. First, the use of an efficient NAD(P)H regeneration system to move reactions that are close to thermodynamic equilibrium forward. Further, the ptdh*-based NAD(P)H regeneration did not evolve CO₂ during cofactor regeneration, improving the carbon negativity of the process. Second, elucidation of the relationship between linear DNA concentrations in the CFE to concentrations of the Module 2 genes expressed. This relationship allowed us to calculate an optimal Module 2 gene ratio leading to a 33-fold improvement in CH₂-THF-to-serine and glycine conversion when compared to the unoptimized Module 2 catalyst. Importantly, although the Module 2 gene ratios were determined when each gene was expressed independently in the CFE, the ratios identified were successful at pointing towards ratios to be used when all 10-genes were expressed simultaneously.

A constraint of the current CFE-based biocatalyst is the lack of ATP recycling, which could be limiting higher conversion rates. ATP is not only used by the pathway but likely by the endogenous CFE metabolism as well. Further improvements to the multi-enzyme biocatalyst could come from 1) introduction of an ATP recycling systems, 2) elucidation of the relationship between linear DNA concentration to concentrations of shmt to pull glycine to serine, 3) reducing the NADPH competition by endogenous CFE reactions, or 4) controlling the timing and expression levels of the 10 pathway genes to achieve optimized enzyme stoichiometries (Kruyer, et al., “Membrane Augmented Cell-Free Systems: A New Frontier in Biotechnology,” ACS Synth Biol 10:670-681 (2021)).

In the background of the CFE-based biocatalyst there are traces of endogenous CFE metabolism that in this specific work may be siphoning some of the glycine and serine synthesized as well as NAD(P)H generated. Further CFE-based biocatalyst dilution should decrease deviation of these metabolites and potentially lead to greater serine amounts. Additionally, competing reactions could be knocked out in the strains used to prepare the lysate (Rasor, et al., “Toward Sustainable, Cell-free Biomanufacturing,” Curr Opin Biotech, 69:136-144, (2021)) or by direct intervention with small molecule or peptide inhibitors. If thermophilic enzymes for a desired pathway can be expressed in CFE (Kruglikov et al., “Proteins from Thermophilic Thermus thermophilus Often Do Not Fold Correctly in a Mesophilic Expression System Such as Escherichia coli,” ACS Omega, 7:37797-37806 (2022)), then heat denaturation could eliminate competition from background reactions present in mesophilic E. coli lysate. Finally, in this work all pathway enzymes are generated at the same time. In the future, controlling the timing and expression levels of pathway genes could be important for achieving optimized enzyme stoichiometries for multi-step biosynthetic pathways (Kruyer, et al., “Membrane Augmented Cell-Free Systems: A New Frontier in Biotechnology,” ACS Synth Biol 10:670-681 (2021)). Looking ahead, data-driven modeling could help identify metabolic engineering strategies most likely to improve production.

What is claimed is:

1. A method of converting formate to a desired compound comprising:
providing a biocatalyst and formate to form a reaction mixture; and
reacting at least the biocatalyst with formate to produce a first reaction product.

2. The method of claim 1, wherein the biocatalyst comprises:
an unpurified mixture of biosynthetic pathway enzymes

3. The method of claim 2, further comprising forming the unpurified mixture of biosynthetic pathway enzymes by a process, comprising:
forming a mixture comprising:
a cell lysate, one or more biosynthetic pathway genes, one or more cofactors, and one or more energy molecules; and
agitating the mixture to allow cell-free expression of the biosynthetic pathway genes to produce the unpurified mixture of biosynthetic pathway enzymes.

4. The method of claim 2, wherein the unpurified mixture of biosynthetic pathway enzymes comprises one or more enzymes selected from the group consisting of formate-tetrahydrofolate ligase (ftl) (SEQ ID NO: 1), methenyltetrahydrofolate cyclohydrolase (fch) (SEQ ID NO: 2), methylenetetrahydrofolate dehydrogenase (mtdA) (SEQ ID NO: 3), glycine cleavage system H protein (gcvH) (SEQ ID NO: 4), glycine cleavage system L protein (gcvL) (SEQ ID NO: 5), glycine cleavage system P protein (gcvP) (SEQ ID NO: 6), glycine cleavage system T protein (gcvT) (SEQ ID NO: 7), lipoate-protein ligase (lplA) (SEQ ID NO: 8), serine hydroxymethyltransferase (shmt) (SEQ ID NO: 9), phosphonate dehydrogenase mutant (ptdh) (SEQ ID NO: 10), formate dehydrogenase (fdh) (SEQ ID NO: 11 or SEQ ID NO:13), and formate dehydrogenase mutant (fdh*) (SEQ ID NO:12).

5. The method of claim 2, wherein the mixture of biosynthetic pathway enzymes are selected from the group consisting of formate-tetrahydrofolate ligase (ftl) (SEQ ID NO: 1), methenyltetrahydrofolate cyclohydrolase (fch) (SEQ ID NO: 2), methylenetetrahydrofolate dehydrogenase (mtdA) (SEQ ID NO: 3), glycine cleavage system H protein (gcvH) (SEQ ID NO: 4), glycine cleavage system L protein (gcvL) (SEQ ID NO: 5), glycine cleavage system P protein (gcvP) (SEQ ID NO: 6), glycine cleavage system T protein (gcvT) (SEQ ID NO: 7), lipoate-protein ligase (lplA) (SEQ ID NO: 8), serine hydroxymethyltransferase (shmt) (SEQ ID NO: 9), phosphonate dehydrogenase mutant (ptdh) (SEQ ID NO: 10), formate dehydrogenase (fdh) (SEQ ID NO: 11 or SEQ ID NO: 13), formate dehydrogenase mutant (fdh*) (SEQ ID NO: 12).

6. The method of claim 1, wherein the reaction mixture further comprises one or more cofactors and/or one or more energy molecules.

7. The method of claim 1, wherein the reaction mixture further comprises NH₃ and bicarbonate, the method further comprising:
reacting at least the biocatalyst with the NH₃, the bicarbonate, and the first reaction product to produce a second reaction product.

8. The method of claim 7 further comprising:
reacting at least the biocatalyst with the first reaction product and the second reaction product to produce a third reaction product.

9. The method of claim 1, wherein the biocatalyst is in a diluted form.

10. The method of claim 1, wherein the first reaction product is 5,10-methylenetetrahydrofolate.

11. The method of claim 7, wherein the second reaction product is glycine.

12. The method of claim 8, wherein the third reaction product is serine.

13. The method of claim 3, wherein the one or more energy molecules is selected from the group consisting of adenosine triphosphate (ATP), guanosine triphosphate (GTP), cytidine triphosphate (CTP), and uridine triphosphate (UTP).

14. The method of claim 3, wherein the one or more cofactors is selected from the group consisting of NADH, NADPH, or pyridoxal phosphate (PLP), α-lipoic acid, 1,4-dithiothreitol (DTT), tetrahydrofolate, H₂NaPO₄.

15. The method of claim 3, wherein the cell lysate is an E. coli lysate.

16. The method of claim 3, wherein the biosynthetic pathway genes are expressed from one or more plasmids.

17. The method of claim 3, wherein the biosynthetic pathway genes are expressed from linear DNA.

18. The method of claim 3, wherein the biosynthetic pathway genes are expressed from a combination of one or more plasmids and linear DNA.

19. The method of claim 1, wherein the formate is produced from the reduction of carbon dioxide.

20. The method of claim 8 further comprising:
reacting at least the biocatalyst with the third reaction product to produce a fourth reaction product, wherein the fourth reaction product is pyruvate.


SEQUENCE LISTING (36 sequences)

SEQ ID NO: 1 - protein, 557 aa, synthetic construct
  source 1..557: note = formate-tetrahydrofolate ligase
MPSDIEIARAATLKPIAQVAEKLGIPDEALHNYGKHIAKIDHDFIASLEGKPEGKLVLVTAISPTPAGEGKTTTTVGLGDALNRIGKRAVMCLREPSLGPCFGMKGGAAGGGKAQVVPMEQINLHFTGDFHAITSAHSLAAALIDNHIYWANELNIDVRRIHWRRVVDMNDRALRAINQSLGGVANGFPREDGFDITVASEVMAVFCLAKNLADLEERLGRIVIAETRDRKPVTLADVKATGAMTVLLKDALQPNLVQTLEGNPALIHGGPFANIAHGCNSVIATRTGLRLADYTVTEAGFGADLGAEKFIDIKCRQTGLKPSAVVIVATIRALKMHGGVNKKDLQAENLDALEKGFANLERHVHNVRSFGLPVVVGVNHFFQDTDAEHVRLKELCRDRLQVEAITCKHWAEGGAGAEALAQAVVKLAEGEQKPLTFAYETETKITDKIKAIATKLYGAADIQIESKAATKLAGFEKDGYGKLPVCMAKTQYSFSTDPTLMGAPSGHLVSVRDVRLSAGAGFVVVICGEIMTMPGLPKVPAADTIRLDANGQIDGLF

SEQ ID NO: 2 - protein, 208 aa, synthetic construct
  source 1..208: note = methenyl-tetrahydrofolate cyclohydrolase
MAGNETIETFLDGLASSAPTPGGGGAAAISGAMGAALVSMVCNLTIGKKKYVEVEADLKQVLEKSEGLRRTLTGMIADDVEAFDAVMGAYGLPKNTDEEKAARAAKIQEALKTATDVPLACCRVCREVIDLAEIVAEKGNLNVISDAGVAVLSAYAGLRSAALNVYVNAKGLDDRAFAEERLKELEGLLAEAGALNERIYETVKSKVN

SEQ ID NO: 3 - protein, 288 aa, synthetic construct
  source 1..288: note = methylenetetrahydrofolate dehydrogenase
MSKKLLFQFDTDATPSVFDVVVGYDGGADHITGYGNVTPDNVGAYVDGTIYTRGGKEKQSTAIFVGGGDMAAGERVFEAVKKRFFGPFRVSCMLDSNGSNTTAAAGVALVVKAAGGSVKGKKAVVLAGTGPVGMRSAALLAGEGAEVVLCGRKLDKAQAAADSVNKRFKVNVTAAETADDASRAEAVKGAHFVFTAGAIGLELLPQAAWQNESSIEIVADYNAQPPLGIGGIDATDKGKEYGGKRAFGALGIGGLKLKLHRACIAKLFESSEGVFDAEEIYKLAKEMA

SEQ ID NO: 4 - protein, 129 aa, Escherichia coli
  source 1..129: note = glycine cleavage system (gcv) H protein
MSNVPAELKYSKEHEWLRKEADGTYTVGITEHAQELLGDMVFVDLPEVGATVSAGDDCAVAESVKAASDIYAPVSGEIVAVNDALSDSPELVNSEPYAGGWIFKIKASDESELESLLDATAYEALLEDE

SEQ ID NO: 5 - protein, 474 aa, Escherichia coli [sequence omitted]

SEQ ID NO: 6 - protein, 957 aa, Escherichia coli
  source 1..957: note = glycine cleavage system (gcv) P protein
MTQTLSQLENSGAFIERHIGPDAAQQQEMLNAVGAQSLNALTGQIVPKDIQLATPPQVGAPATEYAALAELKAIASRNKRFTSYIGMGYTAVQLPPVILRNMLENPGWYTAYTPYQPEVSQGRLEALLNFQQVTLDLTGLDMASASLLDEATAAAEAMAMAKRVSKLKNANRFFVASDVHPQTLDVVRTRAETFGFEVIVDDAQKVLDHQDVFGVLLQQVGTTGEIHDYTALISELKSRKIVVSVAADIMALVLLTAPGKQGADIVFGSAQRFGVPMGYGGPHAAFFAAKDEYKRSMPGRIIGVSKDAAGNTALRMAMQTREQHIRREKANSNICTSQVLLANIASLYAVYHGPVGLKRIANRIHRLTDILAAGLQQKGLKLRHAHYFDTLCVEVADKAGVLTRAEAAEINLRSDILNAVGITLDETTTRENVMQLFNVLLGDNHGLDIDTLDKDVAHDSRSIQPAMLRDDEILTHPVFNRYHSETEMMRYMHSLERKDLALNQAMIPLGSCTMKLNAAAEMIPITWPEFAELHPFCPPEQAEGYQQMIAQLADWLVKLTGYDAVCMQPNSGAQGEYAGLLAIRHYHESRNEGHRDICLIPASAHGTNPASAHMAGMQVVVVACDKNGNIDLTDLRAKAEQAGDNLSCIMVTYPSTHGVYEETIREVCEVVHQFGGQVYLDGANMNAQVGITSPGFIGADVSHLNLHKTFCIPHGGGGPGMGPIGVKAHLAPFVPGHSVVQIEGMLTRQGAVSAAPFGSASILPISWMYIRMMGAEGLKKASQVAILNANYIASRLQDAFPVLYTGRDGRVAHECILDIRPLKEETGISELDIAKRLIDYGFHAPTMSFPVAGTLMVEPTESESKVELDRFIDAMLAIRAEIDQVKAGVWPLEDNPLVNAPHIQSELVAEWAHPYSREVAVFPAGVADKYWPTVKRLDDVYGDRNLFCSCVPISEYQ

SEQ ID NO: 7 - protein, 364 aa, Escherichia coli
  source 1..364: note = glycine cleavage system (gcv) T protein
MAQQTPLYEQHTLCGARMVDFHGWMMPLHYGSQIDEHHAVRTDAGMFDVSHMTIVDLRGSRTREFLRYLLANDVAKLTKSGKALYSGMLNASGGVIDDLIVYYFTEDFFRLVVNSATREKDLSWITQHAEPFGIEITVRDDLSMIAVQGPNAQAKAATLFNDAQRQAVEGMKPFFGVQAGDLFIATTGYTGEAGYEIALPNEKAADFWRALVEAGVKPCGLGARDTLRLEAGMNLYGQEMDETISPLAANMGWTIAWEPADRDFIGREALEVQREHGTEKLVGLVMTEKGVLRNELPVRFTDAQGNQHEGIITSGTFSPTLGYSIALARVPEGIGETAIVQIRNREMPVKVTKPVFVRNGKAVA

SEQ ID NO: 8 - protein, 338 aa, Escherichia coli
  source 1..338: note = lipoate-protein ligase
MSTLRLLISDSYDPWFNLAVEECIFRQMPATQRVLFLWRNADTVVIGRAQNPWKECNTRRMEEDNVRLARRSSGGGAVFHDLGNTCFTFMAGKPEYDKTISTSIVLNALNALGVSAEASGRNDLVVKTVEGDRKVSGSAYRETKDRGFHHGTLLLNADLSRLANYLNPDKKKLAAKGITSVRSRVTNLTELLPGITHEQVCEAITEAFFAHYGERVEAEIISPNKTPDLPNFAETFARQSSWEWNFGQAPAFSHLLDERFTWGGVELHFDVEKGHITRAQVFTDSLNPAPLEALAGRLQGCLYRADMLQQECEALLVDFPEQEKELRELSAWMAGAVR

SEQ ID NO: 9 - protein, 417 aa, Escherichia coli
  source 1..417: note = serine hydroxymethyltransferase
MLKREMNIADYDAELWQAMEQEKVRQEEHIELIASENYTSPRVMQAQGSQLTNKYAEGYPGKRYYGGCEYVDIVEQLAIDRAKELFGADYANVQPHSGSQANFAVYTALLEPGDTVLGMNLAHGGHLTHGSPVNFSGKLYNIVPYGIDATGHIDYADLEKQAKEHKPKMIIGGFSAYSGVVDWAKMREIADSIGAYLFVDMAHVAGLVAAGVYPNPVPHAHVVTTTTHKTLAGPRGGLILAKGGSEELYKKLNSAVFPGGQGGPLMHVIAGKAVALKEAMEPEFKTYQQQVAKNAKAMVEVFLERGYKVVSGGTDNHLFLVDLVDKNLTGKEADAALGRANITVNKNSVPNDPKSPFVTSGIRVGTPAITRRGFKEAEAKELAGWMCDVLDSINDEAVIERIKGKVLDICARYPVYA

SEQ ID NO: 10 - protein, 336 aa, synthetic construct
  source 1..336: note = phosphonate dehydrogenase mutant
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHAAKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKAEPAAC

SEQ ID NO: 11 - protein, 309 aa, synthetic construct
  source 1..309: note = formate dehydrogenase
MAMRQAAKATIRACSSSSSSGYFARRQFNASSGDSKKIVGVFYKANEYATKNPNFLGCVENALGIRDWLESQGHQYIVTDDKEGPDCELEKHIPDLHVLISTPFHPAYVTAERIKKAKNLKLLLTAGIGSDHIDLQAAAAAGLTVAEVTGSNVVSVAEDELMRILILMRNFVPGYNQVVKGEWNVAGIAYRAYDLEGKTIGTVGAGRIGKLLLQRLKPFGCNLLYHDRLQMAPELEKETGAKFVEDLNEMLPKCDVIVINMPLTEKTRGMFNKELIGKLKKGVLIVNNARGAIMERQAVVDAVESGHIG

SEQ ID NO: 12 - protein, 307 aa, synthetic construct
  source 1..307: note = formate dehydrogenase mutant
MRQAAKATIRACSSSSSSGYFARRQFNASSGDSKKIVGVFYKANEYATKNPNFLGCVENALGIRDWLESQGHQYIVTDDKEGPDCELEKHIPDLHVLISTPFHPAYVTAERIKKAKNLKLLLTAGIGSDHIDLQAAAAAGLTVAEVTGSNVVSVAEDELMRILILMRNFVPGYNQVVKGEWNVAGIAYRAYDLEGKTIGTVGAGRIGKLLLQRLKPFGCNLLYHQRHQMAPELEKETGAKFVEDLNEMLPKCDVIVINMPLTEKTRGMFNKELIGKLKKGVLIVNNARGAIMERQAVVDAVESGHIG

SEQ ID NO: 13 - protein, 364 aa, Candida boidinii
  source 1..364: note = formate dehydrogenase
MKIVLVLYDAGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYITKAYGKHDKK

SEQ ID NO: 14 - DNA, 1674 bp, synthetic construct
  source 1..1674: mol_type = other DNA; note = formate-THF ligase
atgccgagcgatattgaaattgcacgcgctgctactctgaaaccgattgcgcaagttgcggagaaactgggtattccggacgaggctcttcataattatggcaaacatatcgctaaaatcgaccatgactttattgcttctcttgagggtaaaccagagggcaaacttgttctggttactgctatttcgccgactccagctggcgagggcaaaactactactactgttggtctgggcgatgctctcaaccgcattggcaaacgtgctgttatgtgtctgcgcgagccctctctcggcccctgttttggcatgaaaggcggcgctgctggtggcggcaaagctcaggttgttccgatggagcagattaatctgcacttcaccggcgattttcacgctattacttctgctcactctctcgctgctgctctgattgataaccatatttattgggctaacgaactgaatattgacgttcgccgcattcattggcgccgcgttgttgatatgaacgatcgggctctgcgcgctattaatcagtctctcggcggcgttgctaatggctttccgcgcgaggatgggtttgacattactgttgcttctgaggttatggctgtgttttgcctcgccaagaatctggctgatcttgaggagcggctcggccgcattgttattgcagaaactcgcgatcgcaaaccggttactctggctgatgttaaagctactggcgctatgactgttctgctcaaggatgctcttcagccgaatctcgtgcagactctggagggcaacccggctctgattcacggcggcccgtttgctaacattgctcatggctgtaactcggttattgctactcgcactggcctgcggctcgctgactatactgttactgaggctggctttggcgctgatctcggcgctgagaaattcattgatattaaatgtcgccagactggcctcaagccctctgctgttgttattgttgctacgattcgcgctctcaaaatgcatggcggcgttaacaagaaagatctccaggctgagaatctggatgcgctggagaaaggttttgcaaatcttgagcgccatgttcacaatgttcgctcttttggcctgccggttgttgttggtgttaaccacttctttcaggatactgatgctgagcatgttcggttgaaagaactgtgccgcgatcggcttcaggttgaggctattacttgtaagcattgggctgagggcggcgcaggcgcagaagcactggcacaggcagttgttaaactggctgaaggcgagcagaaaccgctgacttttgcatatgagaccgaaactaagattactgacaagattaaggcaattgctactaaactgtatggtgctgctgatattcagattgagtctaaagccgccactaagctcgctggcttcgagaaagatggctatggtaagctgccggtctgtatggccaagactcaatattcattttctactgatccgactcttatgggcgctccctctggtcatctggtttctgtgcgcgatgttcgcctctctgctggcgctggcttcgttgttgttatttgtggtgagattatgaccatgccgggtctgccgaaggttccagcagcagatactattcgcctcgatgctaacggtcagattgatgggctgttctag

SEQ ID NO: 15 - DNA, 627 bp, synthetic construct
  source 1..627: mol_type = other DNA; note = methenyl-THF cyclohydrolase
atggctggcaatgagactattgaaacattcttggacggcctggcatcatctgctccgactcccggcggcggcggtgcagcagcaatttctggcgcaatgggcgcagcacttgtttctatggtttgcaatcttactattggcaagaagaaatatgttgaggttgaggcagacttaaaacaggttctggagaaatctgaaggcctgcgccgcactctcactggcatgattgcagacgacgttgaagcctttgacgcagttatgggcgcttatgggctgccgaagaatactgacgaagagaaagcagcacgcgcagcaaagattcaagaggcactcaaaactgcaactgacgttccgctcgcatgttgtcgcgtttgtcgcgaggttattgatctggcagagattgttgcagagaaaggcaatctcaatgttatttctgatgcaggcgttgcagtgctctctgcttatgcaggtctgcgctctgctgcacttaatgtctatgtaaatgcaaaaggcctcgacgaccgcgcatttgcagaggagcggcttaaagagctggagggcctactggctgaggcaggtgcactcaatgagcgaatttatgagactgttaaatctaaagtgaattga

SEQ ID NO: 16 - DNA, 867 bp, synthetic construct
  source 1..867: mol_type = other DNA; note = methylene THF dehydrogenase
atgtctaagaaactgctctttcagtttgacactgatgcaactccgtctgtatttgacgttgttgttggctatgacggcggtgcagaccatattactggctatggcaatgttactcccgacaatgttggcgcatatgttgacggcactatttatactcgtggaggcaaagagaaacagtctacagcaatctttgttggcggcggcgacatggcagcaggcgagcgggtatttgaggcagtaaagaagcgtttctttggcccgtttcgcgtttcttgtatgctggattctaatggctctaatactactgcagcagcaggcgttgcactcgttgttaaagcagcaggcggctctgttaaaggcaagaaagcagttgttctcgcaggtactggtccggttggtatgcgctctgcagctctgttagccggcgagggcgcagaggttgttctgtgtgggcgcaaactcgacaaagcacaggcagcagcagattctgttaataaacgcttcaaagttaatgttactgcagcagagactgcagacgacgcatctcgcgcagaggccgtgaaaggcgcacattttgtctttactgcaggtgcaattggccttgaactgctgccgcaggcagcatggcagaatgagtcttctattgaaattgtggccgattataatgcacagccgccgctcggcattggcgggattgatgcaactgacaaaggcaaagaatatggcggaaaacgcgcatttggtgcgctcggcattggcggcttgaaactcaaactgcatcgcgcatgtattgcaaaactgtttgagtcttctgaaggtgtatttgatgcagaggagatttataaactggcaaaagaaatggcatga

SEQ ID NO: 17 - DNA, 390 bp, Escherichia coli
  source 1..390: mol_type = genomic DNA; note = glycine cleavage system gcv H protein
atgagcaacgtaccagcagaactgaaatacagcaaagaacacgaatggctgcgtaaagaagccgacggcacttacaccgttggtattaccgaacatgctcaggagctgttaggcgatatggtgtttgttgacctgccggaagtgggcgcaacggttagcgcgggcgatgactgcgcggttgccgaatcggtaaaagcggcgtcagacatttatgcgccagtaagcggtgaaatcgtggcggtaaacgacgcactgagcgattccccggaactggtgaacagcgaaccgtatgcaggcggctggatctttaaaatcaaagccagcgatgaaagcgaactggaatcactgctggatgcgaccgcatacgaagcattgttagaagacgagtaa

SEQ ID NO: 18 - DNA, 1425 bp, Escherichia coli
  source 1..1425: mol_type = genomic DNA; note = gcv L protein
atgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccgaaccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtaa

SEQ ID NO: 19 - DNA, 2874 bp, Escherichia coli
  source 1..2874: mol_type = genomic DNA; note = gcvP protein
atgacacagacgttaagccagcttgaaaacagcggcgcttttattgaacgccatatcggaccggacgccgcgcaacagcaagaaatgctgaatgccgttggtgcacaatcgttaaacgcgctgaccggccagattgtgccgaaagatattcaacttgcgacaccaccgcaggttggcgcaccggcgaccgaatacgccgcactggcagaactcaaggctattgccagtcgcaataaacgcttcacgtcttacatcggcatgggttacaccgccgtgcagctaccgccggttatcctgcgtaacatgctggaaaatccgggctggtataccgcgtacactccgtatcaacctgaagtctcccagggccgccttgaagcactgctcaacttccagcaggtaacgctggatttgactggactggatatggcctctgcttctcttctggacgaggccaccgctgccgccgaagcaatggcgatggcgaaacgcgtcagcaaactgaaaaatgccaaccgcttcttcgtggcttccgatgtgcatccgcaaacgctggatgtggtccgtactcgtgccgaaacctttggttttgaagtgattgtcgatgacgcgcaaaaagtgctcgaccatcaggacgtcttcggcgtgctgttacagcaggtaggcactaccggtgaaattcacgactacactgcgcttattagcgaactgaaatcacgcaaaattgtggtcagcgttgccgccgatattatggcgctggtgctgttaactgcgccgggtaaacagggcgcggatattgtttttggttcggcgcaacgcttcggcgtgccgatgggctacggtggcccacacgcggcattctttgcggcgaaagatgaatacaaacgctcaatgccgggccgtattatcggtgtatcgaaagatgcagctggcaataccgcgctgcgcatggcgatgcagactcgcgagcaacatatccgccgtgagaaagcgaactccaacatttgtacttcccaggtactgctggcaaacatcgccagcctgtatgccgtttatcacggcccggttggcctgaaacgtatcgctaaccgcattcaccgtctgaccgatatcctggcggcgggcctgcaacaaaaaggtctgaaactgcgccatgcgcactatttcgacaccttgtgtgtggaagtggccgacaaagcgggcgtactgacgcgtgccgaagcggctgaaatcaacctgcgtagcgatattctgaacgcggttgggatcacccttgatgaaacaaccacgcgtgaaaacgtaatgcagcttttcaacgtgctgctgggcgataaccacggcctggacatcgacacgctggacaaagacgtggctcacgacagccgctctatccagcctgcgatgctgcgcgacgacgaaatcctcacccatccggtgtttaatcgctaccacagcgaaaccgaaatgatgcgctatatgcactcgctggagcgtaaagatctggcgctgaatcaggcgatgatcccgctgggttcctgcaccatgaaactgaacgccgccgccgagatgatcccaatcacctggccggaatttgccgaactgcacccgttctgcccgccggagcaggccgaaggttatcagcagatgattgcgcagctggctgactggctggtgaaactgaccggttacgacgccgtttgtatgcagccgaactctggcgcacagggcgaatacgcgggcctgctggcgattcgtcattatcatgaaagccgcaacgaagggcatcgcgatatctgcctgatcccggcttctgcgcacggaactaaccccgcttctgcacatatggcaggaatgcaggtggtggttgtggcgtgtgataaaaacggcaacatcgatctgactgatctgcgcgcgaaagcggaacaggcgggcgataacctctcctgtatcatggtgacttatccttctacccacggcgtgtatgaagaaacgatccgtgaagtgtgtgaagtcgtgcatcagttcggcggtcaggtttaccttgatggcgcgaacatgaacgcccaggttggcatcacctcgccgggctttattggtgcggacgtttcacaccttaacctacataaaactttctgcattccgcacggcggtggtggtccgggtatgggaccgatcggcgtgaaagcgcatttggcaccgtttgtaccgggtcatagcgtggtgcaaatcgaaggcatgttaacccgtcagggcgcggtttctgcggcaccgttcggtagcgcctctatcctgccaatcagctggatgtacatccgcatgatgggcgcagaagggctgaaaaaagcaagccaggtggcaatcctcaacgccaactatattgccagccgcctgcaggatgccttcccggtgctgtataccggtcgcgacggtcgcgtggcgcacgaatgtattctcgatattcgcccgctgaaagaagaaaccggcatcagcgagctggatattgccaagcgcctgatcgactacggtttccacgcgccgacgatgtcgttcccggtggcgggtacgctgatggttgaaccgactgaatctgaaagcaaagtggaactggatcgctttatcgacgcgatgctggctatccgcgcagaaattgaccaggtgaaagccggtgtctggccgctggaagataacccgctggtgaacgcgccgcacattcagagcgaactggtcgccgagtgggcgcatccgtacagccgtgaagttgcggtattcccggcaggtgtggcagacaaatactggccgacagtgaaacgtctggatgatgtttacggcgaccgtaacctgttctgctcctgcgtaccgattagcgaataccagtaa

SEQ ID NO: 20 - DNA, 1095 bp, Escherichia coli
  source 1..1095: mol_type = genomic DNA; note = glycine cleavage sysytem T protein
atggcacaacagactcctttgtacgaacaacacacgctttgcggcgctcgcatggtggatttccacggctggatgatgccgctgcattacggttcgcaaatcgacgaacatcatgcggtacgtaccgatgccggaatgtttgatgtgtcacatatgaccatcgtcgatcttcgcggcagccgcacccgggagtttctgcgttatctgctggcgaacgatgtggcgaagctcaccaaaagcggcaaagccctttactcggggatgttgaatgcctctggcggtgtgatagatgacctcatcgtctactactttactgaagatttcttccgcctcgttgttaactccgccacccgcgaaaaagacctctcctggattacccaacacgctgaacctttcggcatcgaaattaccgttcgtgatgacctttccatgattgccgtgcaagggccgaatgcgcaggcaaaagctgccacactgtttaatgacgcccagcgtcaggcggtggaagggatgaaaccgttctttggcgtgcaggcgggcgatctgtttattgccaccactggttataccggtgaagcgggctatgaaattgcgctgcccaatgaaaaagcggccgatttctggcgtgcgctggtggaagcgggtgttaagccatgtggcttgggcgcgcgtgacacgctgcgtctggaagcgggcatgaatctttatggtcaggagatggacgaaaccatctctcctttagccgccaacatgggctggaccatcgcctgggaaccggcagatcgtgactttatcggtcgtgaagccctggaagtgcagcgtgagcatggtacagaaaaactggttggtctggtgatgaccgaaaaaggcgtgctgcgtaatgaactgccggtacgctttaccgatgcgcagggcaaccagcatgaaggcattatcaccagcggtactttctccccgacgctgggttacagcattgcgctggcgcgcgtgccggaaggtattggcgaaacggcgattgtgcaaattcgcaaccgtgaaatgccggttaaagtgacaaaacctgtttttgtgcgtaacggcaaagccgtcgcgtaa

SEQ ID NO: 21 - DNA, 1017 bp, Escherichia coli
  source 1..1017: mol_type = genomic DNA; note = lipoate protein ligase
atgtccacattacgcctgctcatctctgactcttacgacccgtggtttaacctggcggtggaagagtgtatttttcgccaaatgcccgccacgcagcgcgttctgtttctctggcgcaatgccgacacggtagtaattggtcgcgcgcagaacccgtggaaagagtgtaatacccggcggatggaagaagataacgtccgcctggcgcgacgcagtagcggtggcggtgcagtgttccacgatctcggcaatacctgctttacctttatggctggcaagccggagtacgataaaactatctccacgtcgattgtgctcaatgcgctgaacgcgctcggcgtcagcgccgaagcgtccggacgtaacgatctggtggtgaaaaccgtcgaaggcgaccgcaaagtctcaggctcggcctatcgcgaaaccaaagatcgcggcttccaccacggcaccttgctactcaatgccgacctcagccgcctggcaaactatctcaatccggataaaaagaaactggcggcgaaaggcattacgtcggtacgttcccgcgtgaccaacctcaccgagctgttgccggggatcacccatgagcaggtttgcgaggccataaccgaggcctttttcgcccattatggcgagcgcgtggaagcggaaatcatctccccgaacaaaacgccagacttgccaaacttcgccgaaacctttgcccgccagagtagctgggaatggaacttcggtcaggctccggcattctcgcatctgctggatgaacgctttacctggggcggcgtggaactgcatttcgacgttgaaaaaggccatatcacccgcgcacaggtgtttaccgacagcctcaacccagcgccgctggaagccctcgccggacgactgcaaggctgcctgtaccgcgcagatatgctgcaacaggagtgcgaagcgctgttggttgacttcccggaacaggaaaaagagctacgggagttatcggcatggatggcgggggctgtaaggtag

SEQ ID NO: 22 - DNA, 1254 bp, Escherichia coli
  source 1..1254: mol_type = genomic DNA; note = serine hydroxymethyltransferase
atgttaaagcgtgaaatgaacattgccgattatgatgccgaactgtggcaggctatggagcaggaaaaagtacgtcaggaagagcacatcgaactgatcgcctccgaaaactacaccagcccgcgcgtaatgcaggcgcagggttctcagctgaccaacaaatatgctgaaggttatccgggcaaacgctactacggcggttgcgagtatgttgatatcgttgaacaactggcgatcgatcgtgcgaaagaactgttcggcgctgactacgctaacgtccagccgcactccggctcccaggctaactttgcggtctacaccgcgctgctggaaccaggtgataccgttctgggtatgaacctggcgcatggcggtcacctgactcacggttctccggttaacttctccggtaaactgtacaacatcgttccttacggtatcgatgctaccggtcatatcgactacgccgatctggaaaaacaagccaaagaacacaagccgaaaatgattatcggtggtttctctgcatattccggcgtggtggactgggcgaaaatgcgtgaaatcgctgacagcatcggtgcttacctgttcgttgatatggcgcacgttgcgggcctggttgctgctggcgtctacccgaacccggttcctcatgctcacgttgttactaccaccactcacaaaaccctggcgggtccgcgcggcggcctgatcctggcgaaaggtggtagcgaagagctgtacaaaaaactgaactctgccgttttccctggtggtcagggcggtccgttgatgcacgtaatcgccggtaaagcggttgctctgaaagaagcgatggagcctgagttcaaaacttaccagcagcaggtcgctaaaaacgctaaagcgatggtagaagtgttcctcgagcgcggctacaaagtggtttccggcggcactgataaccacctgttcctggttgatctggttgataaaaacctgaccggtaaagaagcagacgccgctctgggccgtgctaacatcaccgtcaacaaaaacagcgtaccgaacgatccgaagagcccgtttgtgacctccggtattcgtgtaggtactccggcgattacccgtcgcggctttaaagaagccgaagcgaaagaactggctggctggatgtgtgacgtgctggacagcatcaatgatgaagccgttatcgagcgcatcaaaggtaaagttctcgacatctgcgcacgttacccggtttacgcataa

SEQ ID NO: 23 - DNA, 1011 bp, synthetic construct
  source 1..1011: mol_type = other DNA; note = phosphonate dehydrogenase mutant
atgctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggaggcatctgagggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcggcgaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctaggagggtatgcggcggatgtattcgaaatggaagactgggctcgcgcggacaggccacagcagatcgatcctgcgctgctcgcgcatccgaatacgctgttcactccgcacatagggtcggcagtgcgcgcggtgcgactggagattgaacgttgtgcagcgcagaacatcctccaggcattggcaggtgagcgcccaatcaacgctgtgaaccgtctgcccaaggccgagcctgccgcatgttga

SEQ ID NO: 24 - DNA, 930 bp, synthetic construct
  source 1..930: mol_type = other DNA; note = formate dehydrogenase
atggcaatgcgtcaggcagcaaaagcaaccattcgtgcatgtagcagcagcagctcaagcggttattttgcacgtcgtcagtttaatgcaagcagcggtgatagcaaaaagattgttggtgttttctacaaggccaacgaatacgcaaccaaaaatccgaattttctgggttgtgttgaaaatgcactgggtattcgtgattggctggaaagccagggtcatcagtatattgttaccgatgataaagaaggtccggattgcgaactggaaaaacatattccggatctgcatgttctgattagcaccccgtttcatccggcatatgtgaccgcagaacgtattaagaaagccaaaaatctgaaactgctgctgaccgcaggtattggtagcgatcatattgatctgcaggcagcagccgcagcaggtctgaccgttgccgaagttaccggtagcaatgttgttagcgttgcggaagatgaactgatgcgtattctgattctgatgcgcaattttgttccgggttataatcaggttgttaaaggcgaatggaatgttgccggtattgcatatcgtgcatatgatctggaaggtaaaaccattggcaccgttggtgcaggtcgtattggtaaactgctgttacagcgtctgaaaccgtttggttgtaatctgctgtatcatgatcgtctgcagatggcaccggaattagaaaaagaaaccggtgccaaatttgtcgaagatctgaatgaaatgctgccgaaatgtgatgtgattgttattaacatgccgctgaccgagaaaacccgtggcatgtttaacaaagaactgattggcaaactgaaaaagggtgtgctgattgttaataatgcacgtggtgcaattatggaacgtcaggccgttgttgatgcagttgaaagcggtcatattggttga

SEQ ID NO: 25 - DNA, 924 bp, synthetic construct
  source 1..924: mol_type = other DNA; note = formate dehydrogenase mutant
atgcgtcaggcagcaaaagcaaccattcgtgcatgtagcagcagcagctcaagcggttattttgcacgtcgtcagtttaatgcaagcagcggtgatagcaaaaagattgttggtgttttctacaaggccaacgaatacgcaaccaaaaatccgaattttctgggttgtgttgaaaatgcactgggtattcgtgattggctggaaagccagggtcatcagtatattgttaccgatgataaagaaggtccggattgcgaactggaaaaacatattccggatctgcatgttctgattagcaccccgtttcatccggcatatgtgaccgcagaacgtattaagaaagccaaaaatctgaaactgctgctgaccgcaggtattggtagcgatcatattgatctgcaggcagcagccgcagcaggtctgaccgttgccgaagttaccggtagcaatgttgttagcgttgcggaagatgaactgatgcgtattctgattctgatgcgcaattttgttccgggttataatcaggttgttaaaggcgaatggaatgttgccggtattgcatatcgtgcatatgatctggaaggtaaaaccattggcaccgttggtgcaggtcgtattggtaaactgctgttacagcgtctgaaaccgtttggttgtaatctgctgtatcatcagcgtcatcagatggcaccggaattagaaaaagaaaccggtgccaaatttgtcgaagatctgaatgaaatgctgccgaaatgtgatgtgattgttattaacatgccgctgaccgagaaaacccgtggcatgtttaacaaagaactgattggcaaactgaaaaagggtgtgctgattgttaataatgcacgtggtgcaattatggaacgtcaggccgttgttgatgcagttgaaagcggtcatattggttga

SEQ ID NO: 26 - DNA, 1095 bp, Candida boidinii
  source 1..1095: mol_type = genomic DNA; note = O13437
atgaagatcgtcttagtcttatacgacgccggcaagcacgccgccgatgaagagaagttatacggttgcactgaaaacaagttaggtatcgccaactggttaaaggatcaaggccatgaattaatcaccacctccgacaaggaaggcggaaactccgtcttggaccaacatatcccagatgccgatatcatcatcacaactcctttccatcctgcgtacattaccaaggaaagaatcgacaaggccaagaagttgaaattagtcgtcgtcgccggcgtgggttccgaccacatcgacttggactacatcaaccaaaccggcaagaagatctccgtcttggaagtcaccggctccaacgttgtctccgtcgccgaacacgtcctcatgaccatgcttgtcttggtcagaaactttgtcccagcccatgaacaaatcatcaaccacgactgggaagtcgccgccattgcaaaggatgcctacgacatcgaaggtaagaccatcgccaccatcggtgccggtagaatcggttacagagtcttggaaagattagtcccattcaacccaaaggaattattatactacgattaccaagccttaccaaaggacgcagaagaaaaggtcggtgccagaagggtcgaaaacatcgaagaattagtcgcccaagccgacatcgtcaccatcaacgccccattacacgccggtaccaagggtttaatcaacaaggaattattgtctaagttcaagaagggtgcctggttagtcaacaccgccagaggtgccatctgtgtcgcggaggacgtcgccgccgccctggaatccggtcaattaagaggttacggtggtgacgtctggttcccacaacctgccccaaaggaccatccttggagagacatgagaaacaaatacggcgccggcaacgccatgacccctcattactccggtaccaccctggacgcccaaaccagatacgccgaaggtaccaagaacatcttagagtccttcttcaccggtaagtttgactacagaccacaagacatcatcttattaaacggcgaatacatcaccaaggcctatggcaagcacgacaagaagtga

SEQ ID NO: 27 - DNA, 19 bp, synthetic construct
  source 1..19: mol_type = other DNA; note = SC12 primer
gcggtgataatggttgcag

SEQ ID NO: 28 - DNA, 20 bp, synthetic construct
  source 1..20: mol_type = other DNA; note = JS4 primer
actgggttgaaggctctcaa

SEQ ID NO: 29 - DNA, 18 bp, synthetic construct
  source 1..18: mol_type = other DNA; note = RW9 primer
gactatcgcaccatcagc

SEQ ID NO: 30 - DNA, 19 bp, synthetic construct
  source 1..19: mol_type = other DNA; note = RW10 primer
ctgtcctacgagttgcatg

SEQ ID NO: 31 - DNA, 20 bp, synthetic construct
  source 1..20: mol_type = other DNA; note = GH1 primer
gtgatgtcggcgatataggc

SEQ ID NO: 32 - DNA, 16 bp, synthetic construct
  source 1..16: mol_type = other DNA; note = GH2 primer
ctgtccgaccgctttg

SEQ ID NO: 33 - DNA, 16 bp, synthetic construct
  source 1..16: mol_type = other DNA; note = GH3 primer
cgcctgatgcgtgaac

SEQ ID NO: 34 - DNA, 19 bp, synthetic construct
  source 1..19: mol_type = other DNA; note = GH4 primer
gtagcacctgaagtcagcc

SEQ ID NO: 35 - DNA, 55 bp, synthetic construct
  source 1..55: mol_type = other DNA; note = PT70 promoter
tgagctaacaccgtgcgtgttgacaattttacctctggcggtgataatggttgca

SEQ ID NO: 36 - DNA, 19 bp, synthetic construct
  source 1..19: mol_type = other DNA; note = PT3 promoter
attaaccctcactaaaggg